(19) United States
(12) Patent Application Publication
(10) Pub. No.: US 2026/0199481 A1
(43) Pub. Date: Jul. 16, 2026

(54) PROTEIN DEGRADATION TARGETING CHIMERA COMPOUND, COMPOSITION THEREOF, AND USE THEREOF

(71) Applicant: TSINGHUA UNIVERSITY, Beijing City (CN)

(72) Inventors: Jinghong Li, Beijing City (CN); Chunrong Yang, Beijing City (CN); Yuchen Yang, Beijing City (CN)

(21) Appl. No.: 19/135,544

(22) PCT Filed: Dec. 5, 2023
(86) PCT No.: PCT/CN2023/136360

§ 371 Date: Jun. 4, 2025

(30) Foreign Application Priority Data
Dec. 5, 2022 (CN) 202211549110.4

(51) Int. Cl. A61K 47/54; A61P 35/00

(52) U.S. Cl. CPC A61K 47/545; A61P 35/00

(57) ABSTRACT

A protein degradation targeting chimera compound, a composition thereof, and use thereof. The structure of the compound is represented by the following formula (I). The described compound has an X-ray response effect and can solve the problems of systemic toxicity and poor efficacy in tumor treatment after administration of an existing PROTACs drug system.

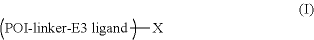

### TECHNICAL FIELD

The disclosure relates to the field of pharmaceuticals, specifically, relates to a protein degradation-targeting chimeric compound and compositions and use thereof.

### BACKGROUND

Proteolysis-Targeting Chimeras (PROTACs) are an emerging technology for protein degradation. PROTACs are heterobifunctional molecules that contain two major functional parts, one end of which binds to the protein of interest (POI) and the other end binds to an E3 ubiquitin ligase. After PROTACs hijack the E3 ubiquitin ligase and bind to the POI, the POI is polyubiquitinated by the ubiquitin-proteasome system (UPS) and degraded. The unique action mechanism of PROTACs not only provides a new approach to the targeted degradation of “undruggable” proteins, but also demonstrates multiple advantages over traditional small-molecule inhibitors such as its unique catalytic properties, lower administration dose and potential to overcome drug resistance. In the past decades, a great deal of research has been devoted to designing new PROTACs to expand the range of target proteins, developing new E3 ligase ligands to broaden the application of PROTACs, and improving the performance of PROTACs from multiple perspectives.

Despite the significant advantages of PROTACs in targeting protein degradation, the potential systemic toxicity of PROTACs caused by undesired off-tissue protein degradation remains an urgent problem. To address this problem, researchers have proposed a series of strategies for the design of stimuli-responsive PROTAC prodrugs. Among them, light-dependent PROTACs (photoPROTACs) prodrugs are the most representative class of strategies, and the feasibility of light-regulated protein degradation has been verified in vitro. Although light-induced protein degradation can be achieved in vitro by different light-responsive chemical designs, these strategies are difficult to be used in deeper tissues due to the poor tissue permeability of UV-visible light. The development of new PROTACs prodrug activation strategies to achieve tissue selectivity, reduce the systemic toxicity, and broaden the application of PROTACs in vivo remains a major challenge in the drug discovery.

Ionizing radiation from X-rays and T-rays has been widely used in radiation therapy. The local radiation therapy can cause DNA damage in tumor cells, which leads to apoptosis and tumor necrosis. In addition, ionizing radiation during radiation therapy induces a series of chemical reactions in cells and living organisms, which in turn activate compounds modified by functional groups. Based on the above background, this disclosure first develops a PROTAC and uses it in combination with radiation therapy to achieve a more effective anti-tumor effect. Further, the present disclosure designs PROTAC prodrugs that are regulated by irradiation and have enhanced radiotherapy effects. The PROTAC prodrug disclosed in this disclosure can be effectively activated in vitro, in tumor cells, and in vivo under irradiation, enabling precise, spatiotemporally controlled targeted protein degradation. Moreover, the released PROTAC drug synergizes with radiotherapy to kill tumor cells, effectively inhibiting tumor growth in mice.

### SUMMARY

An object of the present disclosure is to provide a PROTAC compound.

Another object of the present disclosure is to provide a compound which is a PROTAC drug composition capable of releasing an active ingredient under irradiation.

To achieve these objectives, the present disclosure provides a compound represented by Formula (I), or an isomer, pharmaceutically acceptable salt, or deuterated derivative thereof:

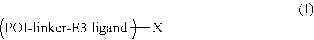

Wherein, the POI is selected from any target protein ligand of interest; the E3 ligand is an E3 ubiquitin ligase ligand; the linker is a functional group linking the target protein ligand and the E3 ubiquitin ligase ligand; and X is any functional molecule responsive to irradiation.

According to some specific embodiments of the present disclosure, wherein said ligand for the target protein may be derived from small molecules, nucleic acid aptamers, various forms of antibodies, and the like.

According to some specific embodiments of the present disclosure, wherein X is structured as shown in formula (I-1) below:

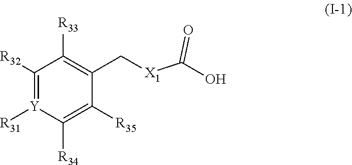

wherein,
R₃₁ is selected from —N₃, H, C1-10 alkyl, or

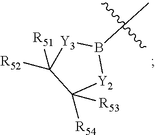

R₃₂ and R₃₄ are each independently selected from H, halogen, or —R₄₁—Y₁—R₄₂;
R₃₃ and R₃₅ are each independently selected from F, Cl, Br, I, or H;
Y₁ is selected from O or S;
R₄₁ is selected from C1-10 alkylene;
R₄₂ is selected from C1-10 alkyl;
R₅₁, R₅₂, R₅₃, and R₅₄ are each independently selected from H or C1-10 alkyl;
Y₂ and Y₃ are each independently selected from O or S;
X₁ is selected from O, S, or NH;
Y is selected from C or N⁺.

According to some specific embodiments of the present disclosure, wherein,
R₃₁ is selected from

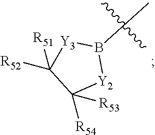

R₃₂, R₃₃, R₃₄ and R₃₅ is selected from H or F, Cl, Br, I;
X₁ is selected from O or S;
Y is C.

According to some specific embodiments of the present disclosure, wherein,
R₃₁ is selected from C1-10 alkyl;
R₃₂, R₃₃, R₃₄ and R₃₅ is selected from H or F, Cl, Br, I;
X₁ is selected from O or S;
Y is selected from N⁺.

According to some specific embodiments of the present disclosure, wherein X is structured as shown in formula (II) below:

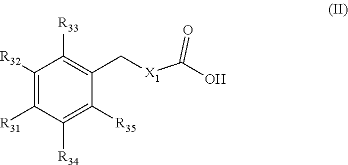

wherein R₃₁ is selected from —N₃ or H;
R₃₂ and R₃₄ are selected from halogen or —R₄₁—Y₁—R₄₂;
R₃₃ and R₃₅ are selected from F, Cl, Br, I or H;
Y₁ is selected from O or S;
R₄₁ is selected from C1-10 alkylidene; R₄₂ is selected from C1-10 alkyl;
X₁ is selected from O, S or NH.

According to some specific embodiments of the present disclosure, wherein X is structured as shown in formula (II-1) or formula (II-2) below:

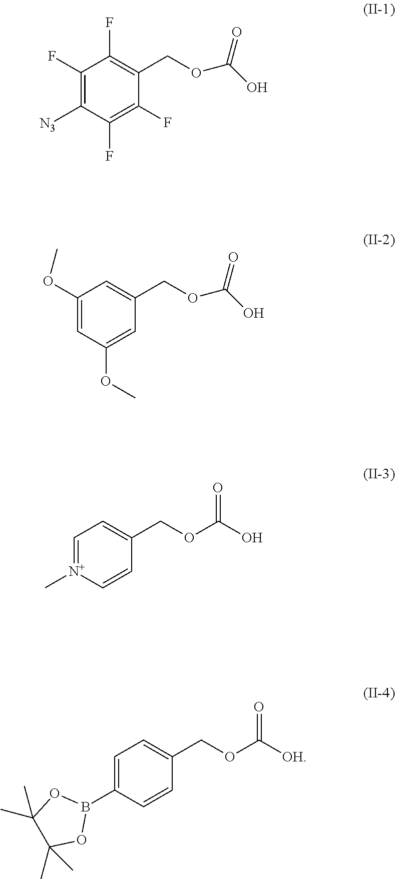 [SMILES: [N-]=[N+]=Nc1c(F)c(F)c(COC(=O)O)c(F)c1F ; COc1cc(COC(=O)O)cc(OC)c1 ; C[n+]1ccc(COC(=O)O)cc1 ; CC1(C)OB(c2ccc(COC(=O)O)cc2)OC1(C)C]

According to some specific embodiments of the present disclosure, wherein the POI ligand is a derivative of BET family protein ligand JQ1.

According to some specific embodiments of the present disclosure, wherein the POI ligand has the structure shown in formula (III) below:

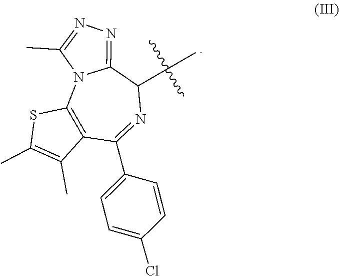 [SMILES: *C1N=C(c2ccc(Cl)cc2)c2c(sc(C)c2C)-n2c(C)nnc21]

According to some specific embodiments of the present disclosure, wherein the linker structure is shown as follows:

—R₁₇—(C═O)ₘ—NH—R₁₁—(R₁₅)ₐ—(NR₂₁)_{b}—X₂—R₁₂-(A)_{c}-R₁₃—(CH₂—O—X₃)_{d}—Y—R₁₄—(R₁₆)ₑ—
R₁₁ and R₁₄ are each independently selected from C1-10 alkylene; R₁₂, R₁₃, and R₁₇ are C1-10 alkylene or a bond;
R₁₅ and R₁₆ are each independently —(C═O)—;
R₂₁ is selected from H or an alkyl group of C1-10;
A is selected from a C4-12 heterocyclylene group or a C5-10 heteroarylene group, said heterocyclylene group or heteroarylene group containing 1-4 heteroatoms independently selected from N, O or S;
X₂, Y are each independently —O—, —S— or a bond;
X₃ is a bond or —CH₂—;
a, b, c, e is 0 or 1; d is 0, 1, 2, 3, 4, 5, 6 or 7; m and n are each independently 0 or 1.

According to some specific embodiments of the present disclosure, wherein said heteroarylene group is selected from one of the following structures:
thienylidene, furanylidene, pyrrolylidene, isothiazolylidene, isoxazolylidene, pyrazolylidene, thiadiazolylidene, oxadiazolylidene, thiadiazolylidene, oxadiazolylidene, tetrazolylidene, triazolylidene, pyridinylidene, pyrimidinylidene, pyridazinylidene, pyrazinylidene, triazinylidene;
said heterocyclylene group is selected from one of the following structures:
azetidine, azolidine, azepane, azocane, azonane, oxetane, oxolane, oxane, oxepane, oxocane, thietane, thiolane, thiane, thiepane, thiocane, diazetidine, diazolidine, diazepane, diazocane, diazonane, dioxetane, dioxolane, dioxane, dioxepane, dioxocane, dithietane, dithiolane, dithiane, dithiepane, dithiocane, and

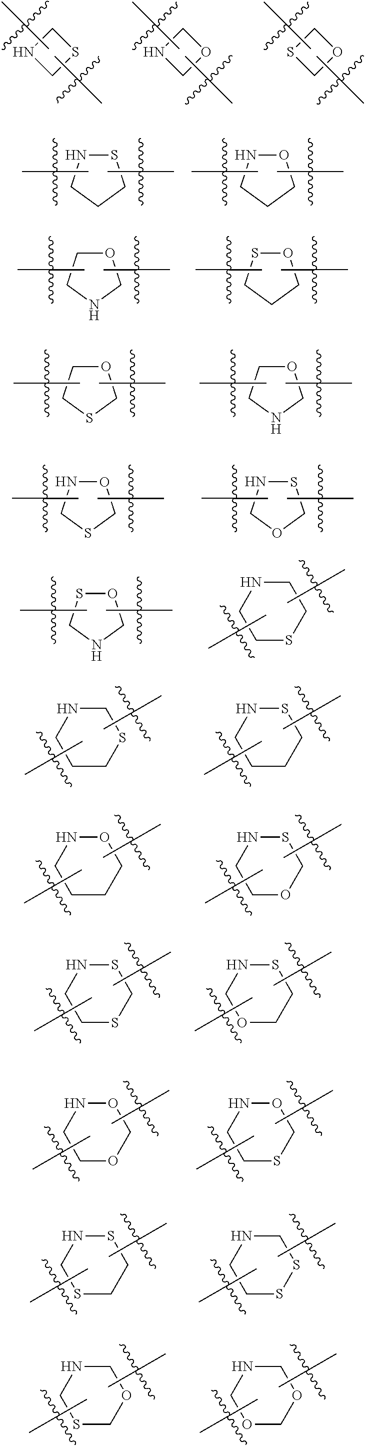

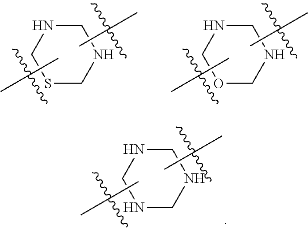

Wherein it is to be understood that the aforesaid heterocyclylene or heterocyclylene group, the sites connected to the POI and the E3 ligand, i.e., can be C atoms or heteroatoms on the heterocyclylene or heterocyclylene groups; and when a C atom or a heteroatom serves as a connecting site, the C atom or the H atoms of the heteroatom are substituted, e.g., when the connecting site is an N atom, the H of the —NH— is substituted, and the —NH— is turned into the

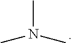

According to some specific embodiments of the present disclosure, wherein said heterocyclylene group is selected from one of the following structures:

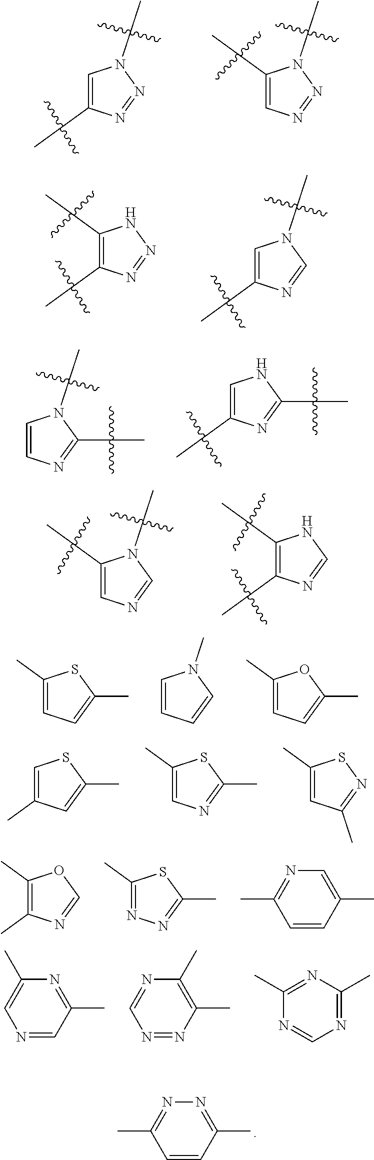

Said heterocyclylene group is selected from one of the following structures:

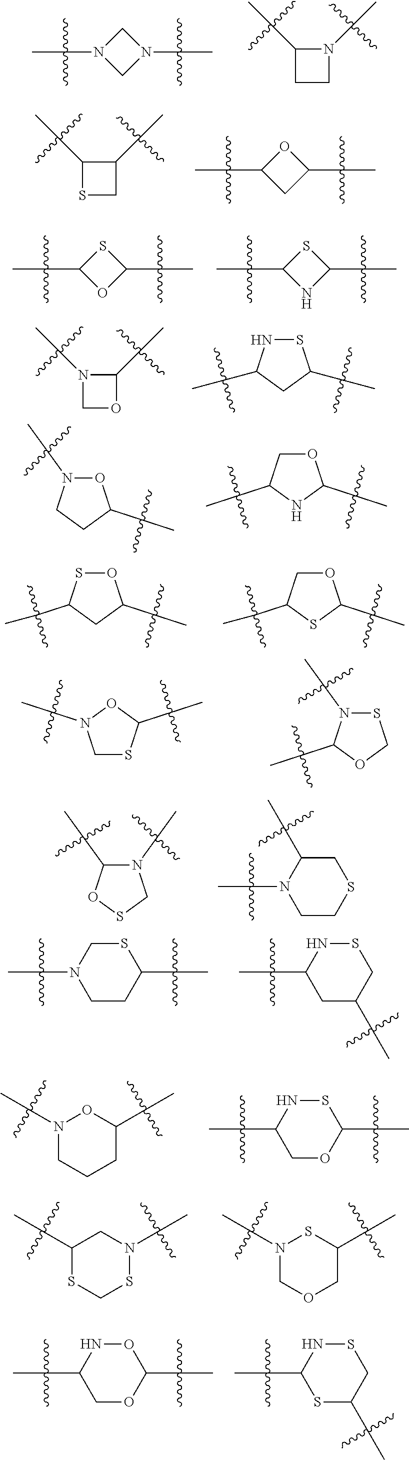

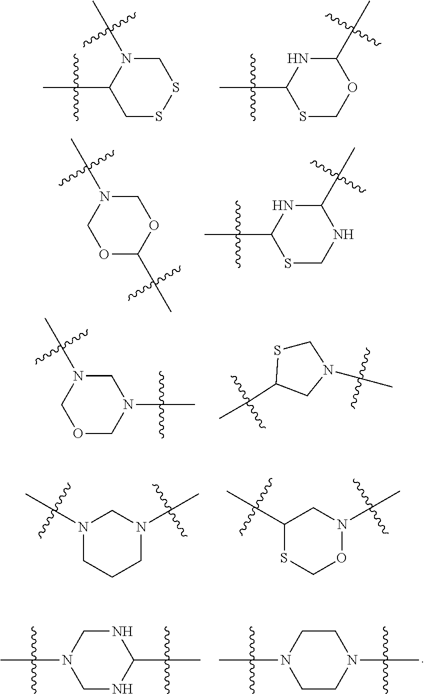

According to some specific embodiments of the present disclosure, wherein the linker structure is selected from one of the following structures:
—(C═O)—NH—R₁₁—(CH₂—O—CH₂)_{d1}—R₁₄—,
—(C═O)—NH—R₁₁—(R₁₅)ₐ—(NR₂₁)—R₁₂—(CH₂—O—CH₂)_{d2}—R₁₄—,
—(C═O)—NH—R₁₁—X—R₁₂-A-R₁₃—(CH₂—O—CH₂)_{d2}—Y—R₁₄—(R₁₆)ₑ—,
—(C═O)—NH—R₁₁—R₁₂—R₁₃—R₁₄—; or
R₁₇—(C═O)—NH—R₁₁—R₁₄—NH—(C═O)—.
wherein R₁, R₁₂, R₁₃, and R₁₄ are each independently selected from alkylidene of C1-4;
R₂₁ is H or alkyl of C1-4; d1 is 1, 2, 3, 4, or 5; and d2 is 0, 1, 2, 3, or 4.

According to some specific embodiments of the present disclosure, wherein the linker structure is selected from one of the following structures:

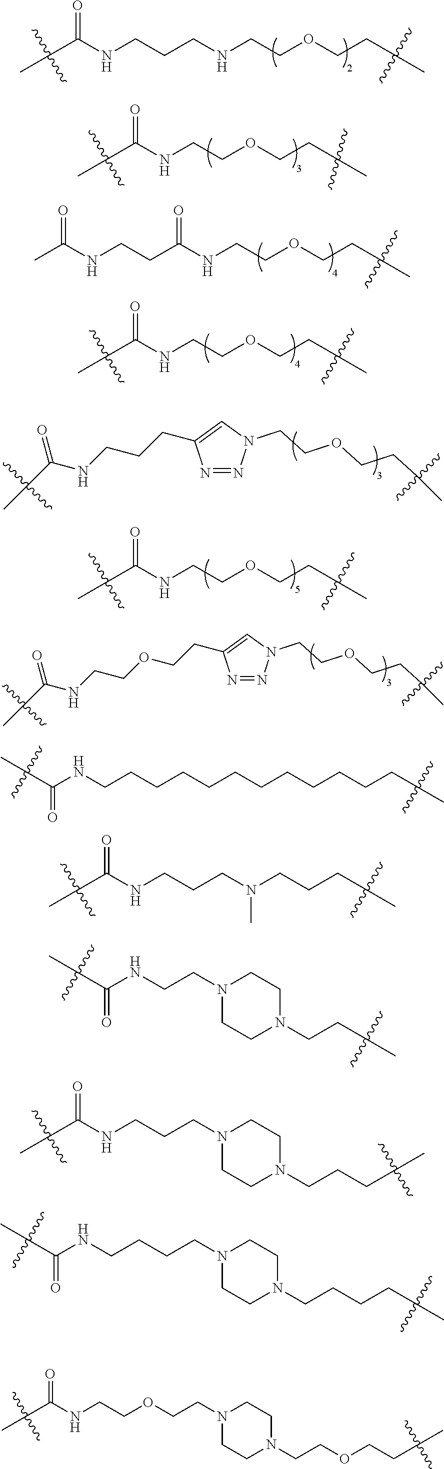

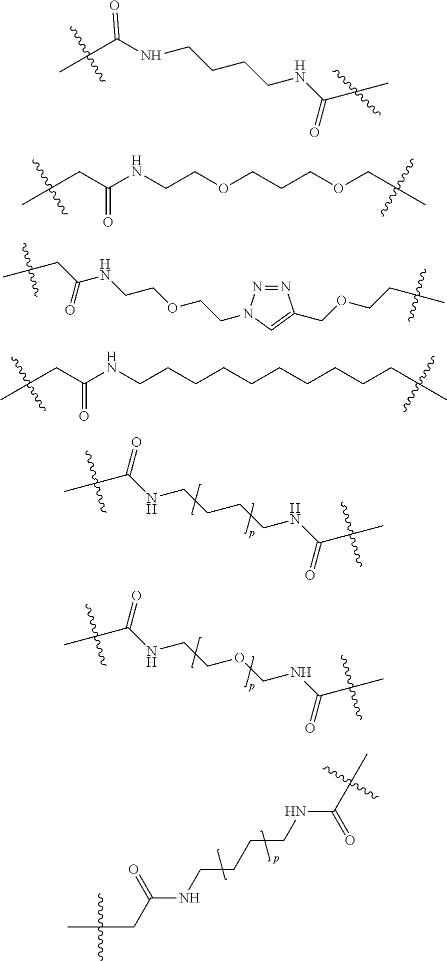

wherein p is 1, 2, 3, 4 or 5.

According to some specific embodiments of the present disclosure, wherein the E3 ligand is selected from the ligands of the E3 ligases of VHL, CRBN, GID4, DCAF2, KLHL41, SPSB4, TRIM7, TRIM9, RNF43 and RNF182.

According to some specific embodiments of the present disclosure, wherein the E3 ligand structure is shown in formula (IV-1) or formula (IV-2) below:

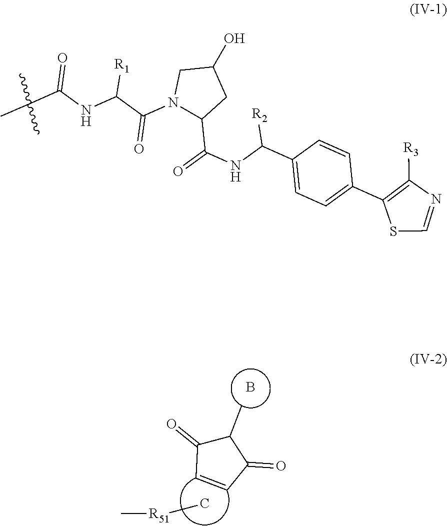

wherein R₁, R₂ and R₃ are each independently selected from an alkyl group of C1-10;
optionally, said alkyl group is further substituted with H, halogen, hydroxyl, carboxyl, amino, cyano, carbonyl or alkyl group of C1-5;
Ring B is a heterocycloalkyl group of C4-10; said heterocycloalkyl group is optionally substituted with 0, 1, 2, 3 or 4=0; said heterocycloalkyl group contains 1, 2, 3 or 4 heteroatoms selected from N, O, and S and contains at least one N atom;
Ring C is an aromatic ring of C6-12; said aromatic ring is optionally substituted with 0, 1, 2, 3, 4, 5 or 6 substituents selected from F, Cl, Br, I, C1-6 alkyl, hydroxy, amino, cyano and nitro;
R₅₁ is selected from —O— or —NH—.

According to some specific embodiments of the present disclosure, wherein X is attached to the E3 ligand structure or to a linker.

According to some specific embodiments of the present disclosure, wherein X is attached to the hydroxyl or amino group of the E3 ligand or linker structure.

Wherein it is to be understood that said amino group to which X is attached, may also be an imino group (—NH—) on the E3 ligand or linker structure.

According to some specific embodiments of the present disclosure, wherein said compounds are structured as shown in the following formulae (V-1), (V-2), (V-3) and (V-4):

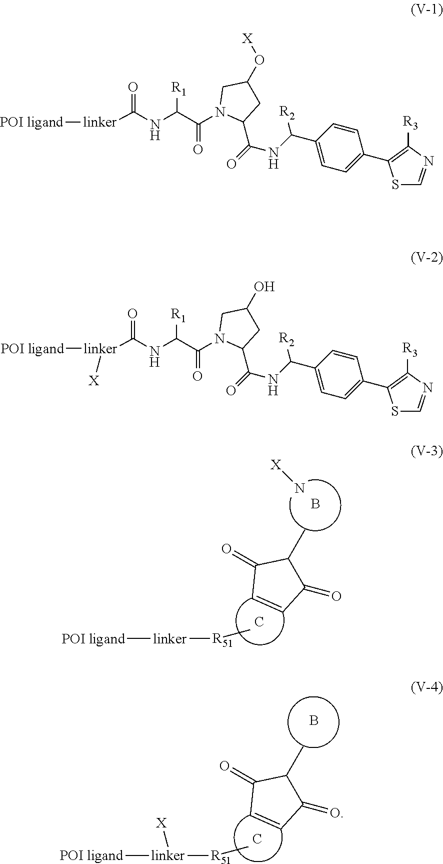

According to some specific embodiments of the present disclosure, wherein R₁, R₂, and R₃ are each independently selected from methyl, ethyl, n-propyl, isopropyl, n-butyl, isobutyl, tert-butyl, n-pentyl, isopentyl, neopentyl, 2-methylbutyl, 3-methylbutyl, n-hexyl, 2-methylpentyl, 3-methylpentyl, 4-methylpentyl, 2-ethylbutyl, or 3-ethylbutyl.

According to some specific embodiments of the present disclosure, wherein the ring B is a heterocycloalkyl group selected from the following structures:

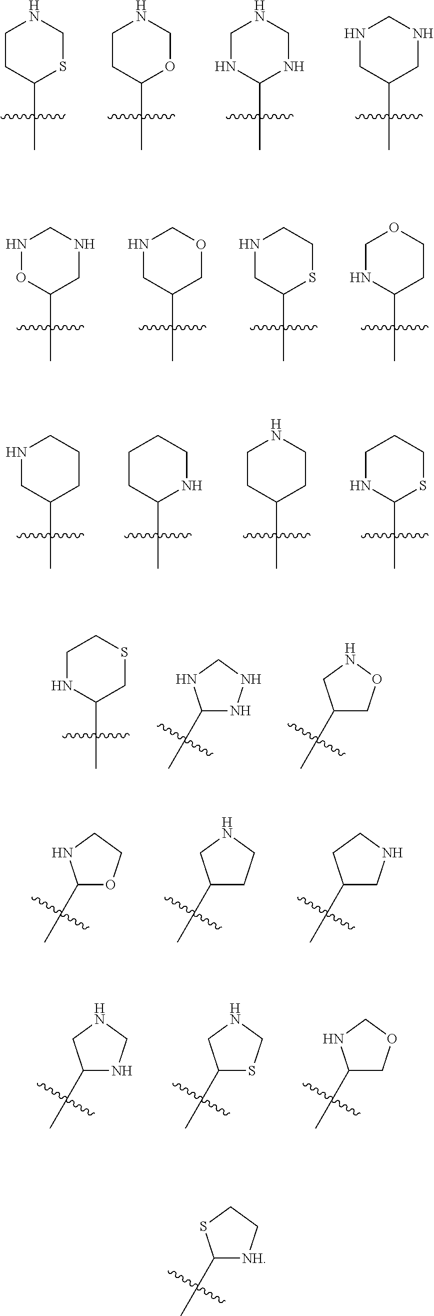

Said heterocycloalkyl group is optionally substituted with 0, 1, 2, 3 or 4=0.

According to some specific embodiments of the present disclosure, wherein ring C is an aromatic ring selected from the following structures:

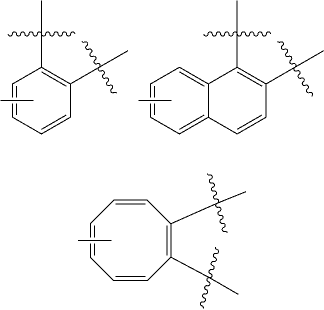

Said aromatic ring is optionally substitute with 0, 1, 2, 3, 4, 5 or 6 substituents selected from F, Cl, Br, I, C1-6 alkyl, hydroxy, amino, cyano and nitro.

According to some specific embodiments of the present disclosure, wherein the ring B is selected from a heterocyclic alkyl group as follows:

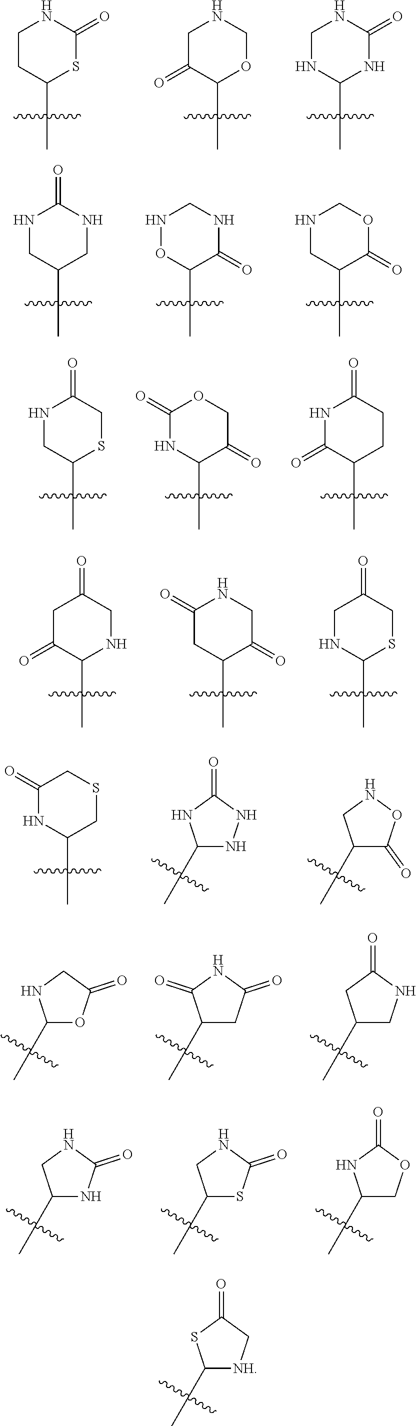

According to some specific embodiments of the present disclosure, wherein ring C is an aromatic ring selected from the following structures:

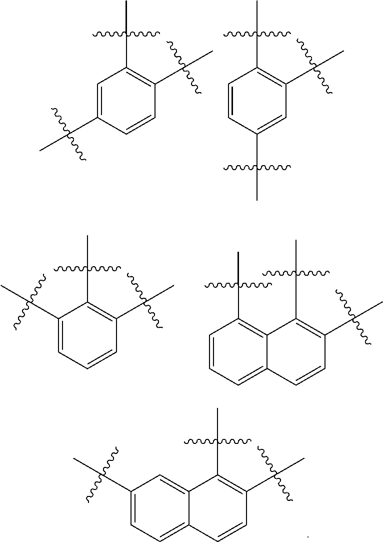 [SMILES: *c1ccc(*)c(*)c1 ; *c1ccc(*)c(*)c1 ; *c1cccc(*)c1* ; *c1ccc2cccc(*)c2c1* ; *c1ccc2ccc(*)c(*)c2c1]

According to some specific embodiments of the present disclosure, wherein said compound is selected from one of the structures shown below:

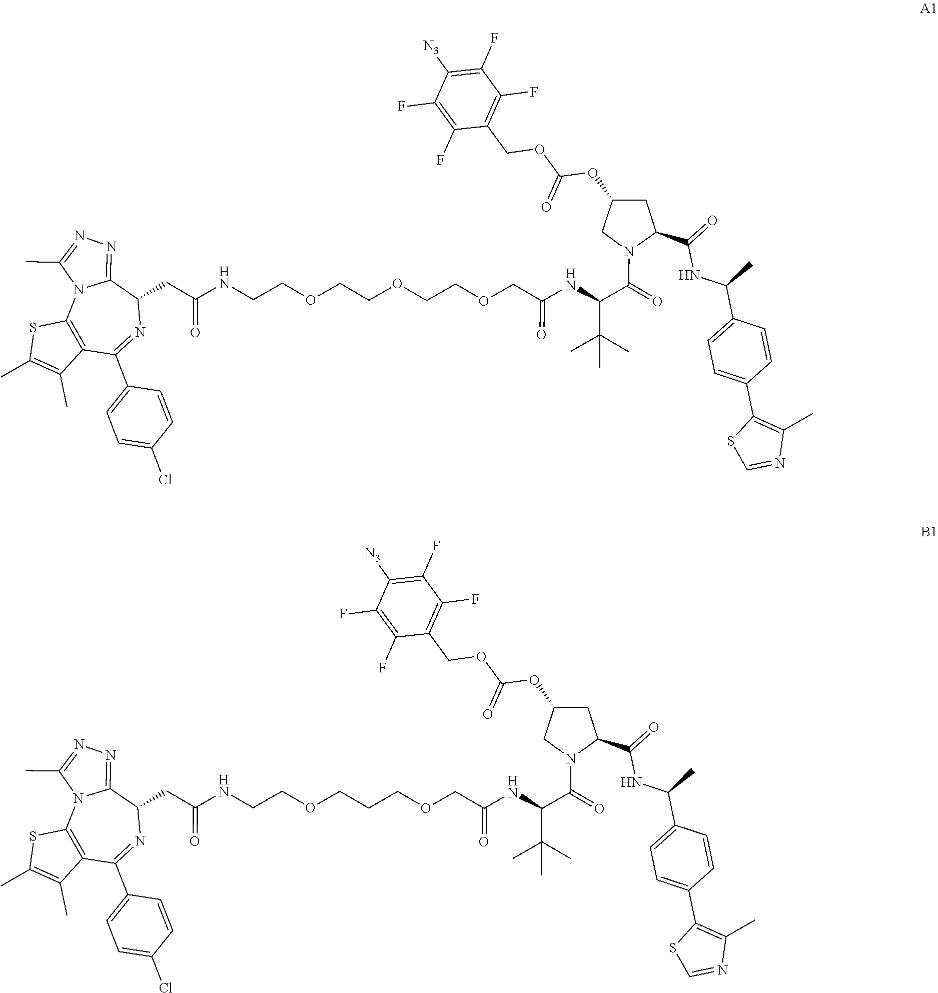

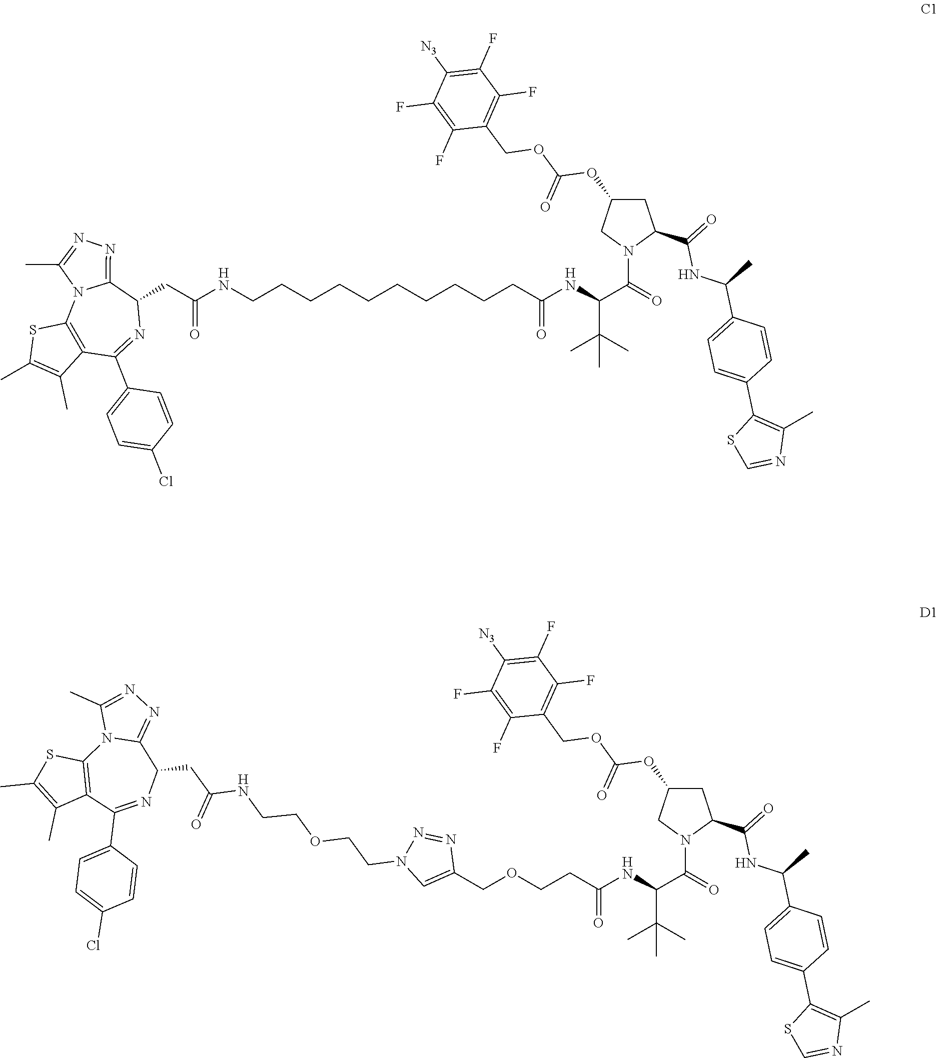

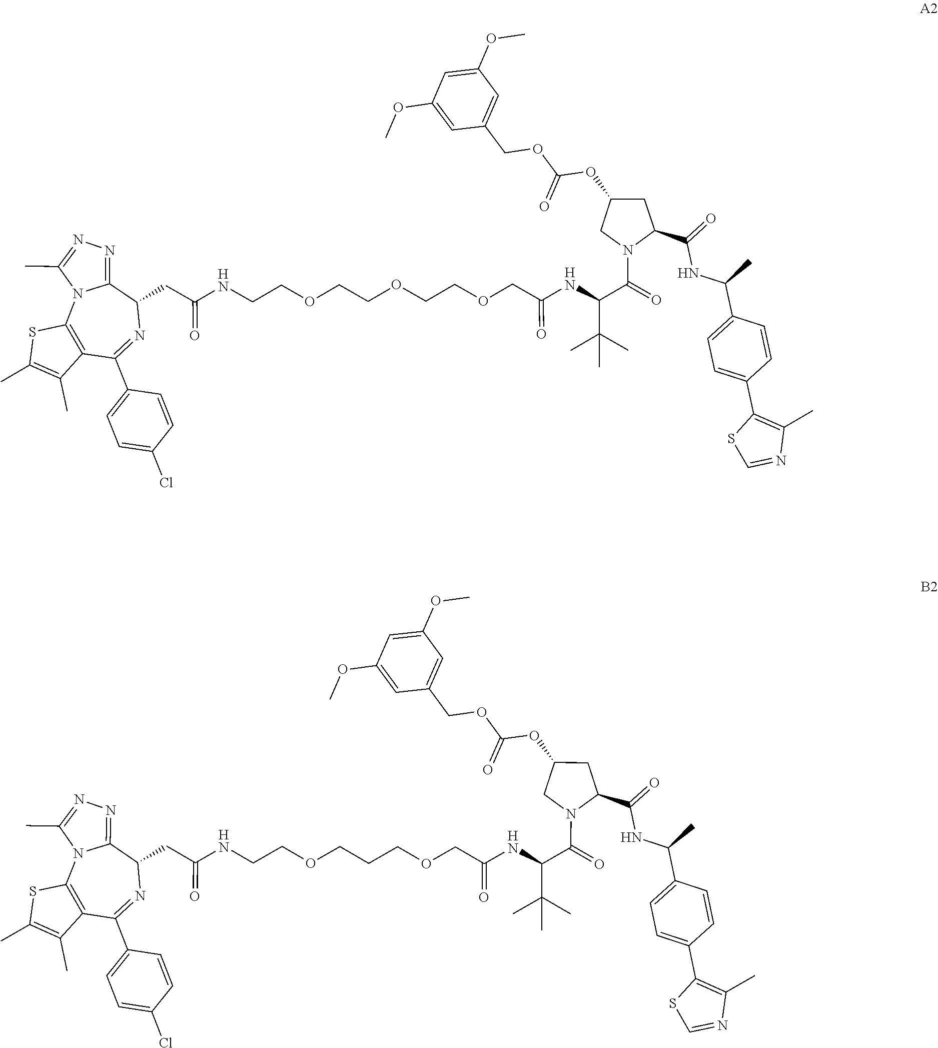

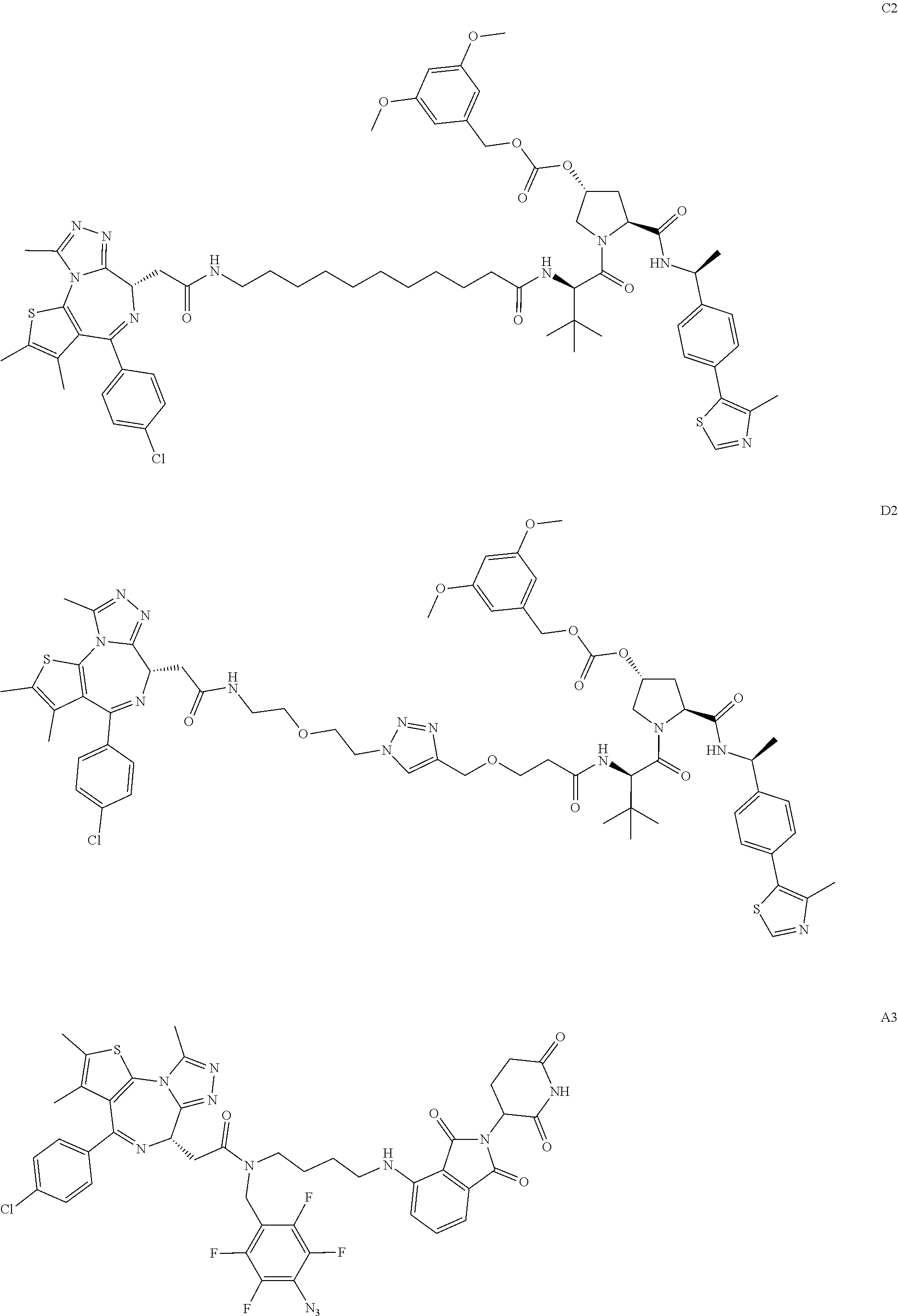

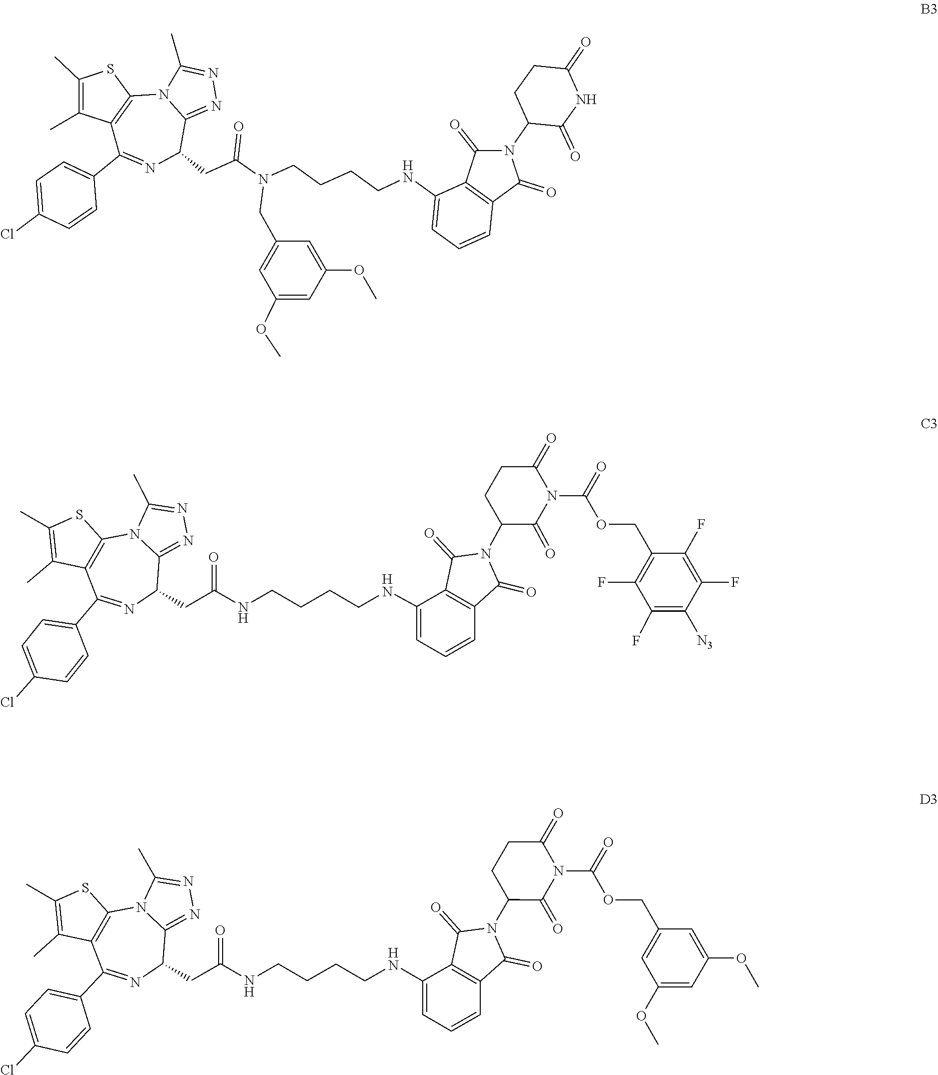 [SMILES: COc1cc(CN(CCCCNc2cccc3c2C(=O)N(C2CCC(=O)NC2=O)C3=O)C(=O)C[C@@H]2N=C(c3ccc(Cl)cc3)c3c(sc(C)c3C)-n3c(C)nnc32)cc(OC)c1 ; Cc1sc2c(c1C)C(c1ccc(Cl)cc1)=N[C@@H](CC(=O)NCCCCNc1cccc3c1C(=O)N(C1CCC(=O)N(C(=O)OCc4c(F)c(F)c(N=[N+]=[N-])c(F)c4F)C1=O)C3=O)c1nnc(C)n1-2 ; COc1cc(COC(=O)N2C(=O)CCC(N3C(=O)c4cccc(NCCCCNC(=O)C[C@@H]5N=C(c6ccc(Cl)cc6)c6c(sc(C)c6C)-n6c(C)nnc65)c4C3=O)C2=O)cc(OC)c1]

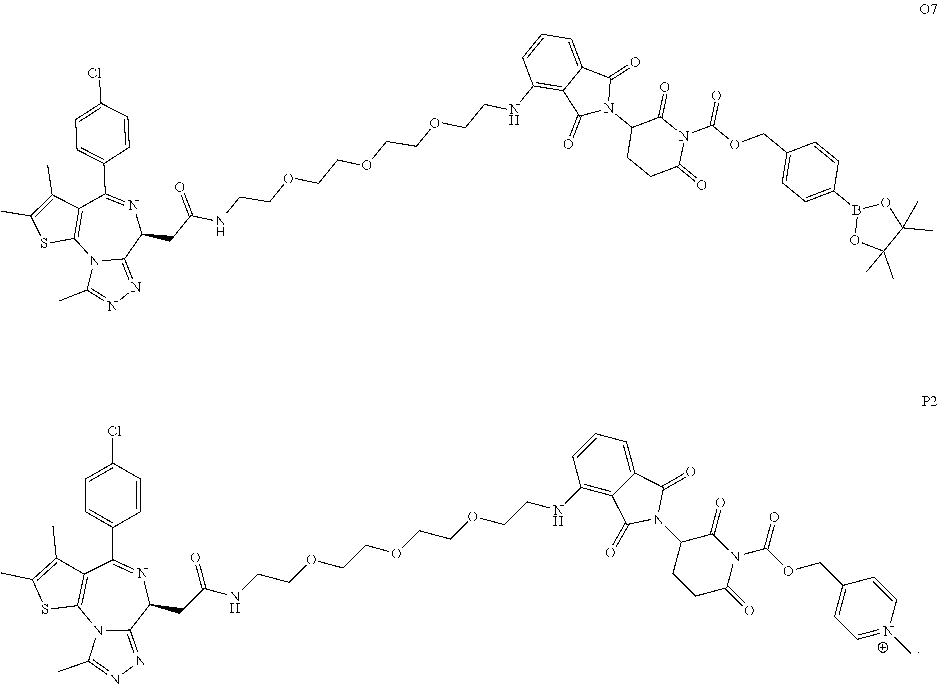

On the other hand, the present disclosure also provides a compound shown in formula (VI) or an isomer thereof or a pharmaceutically acceptable salt or deuterium substituent thereof:

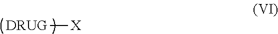

wherein X has the structure as previously described in the present disclosure.

According to some specific embodiments of the present disclosure, wherein DRUG is any PROTAC molecule.

According to some specific embodiments of the present disclosure, wherein DRUG is the following formula (VII)

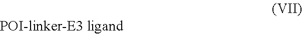

Wherein the POI, linker, and E3 ligand are structured as described anywhere earlier in the present disclosure.

According to some specific embodiments of the present disclosure, wherein X is structured as follows:

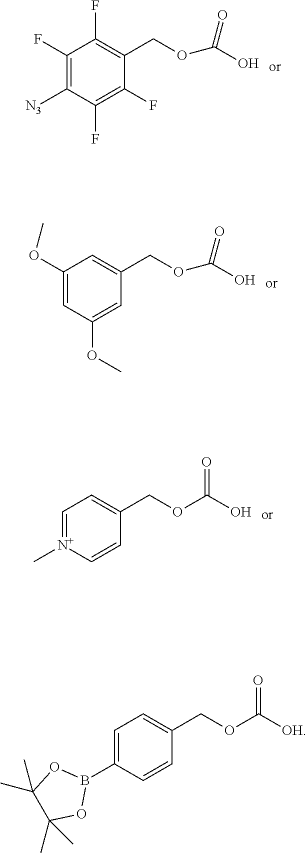 [SMILES: [N-]=[N+]=Nc1c(F)c(F)c(COC(=O)O)c(F)c1F ; COc1cc(COC(=O)O)cc(OC)c1 ; C[n+]1ccc(COC(=O)O)cc1 ; CC1(C)OB(c2ccc(COC(=O)O)cc2)OC1(C)C]

In yet another object, the present disclosure also provides a pharmaceutical composition containing a compound described in any of the present disclosure, including compounds shown in formula (I), formula (V-1), (V-2), (V-3), (V-4), and formula (VI), or isomers, or pharmaceutically acceptable salts or deuterated substituents thereof.

According to some specific embodiments of the present disclosure, wherein said composition further contains a pharmaceutically acceptable carrier or excipient.

The pharmaceutical compositions of the present disclosure can be used to pharmacologically treat a patient in any feasible manner. This includes, but is not limited to: topical administration, oral administration, spray inhalation, rectal administration, nasal administration, vaginal administration, subcutaneous injection, intravenous injection, intramuscular injection, intraperitoneal injection, intracardiac injection, intracranial injection or importation, or administration of medication with the aid of an externally-implanted reservoir, wherein oral, intramuscular or intraventricular modes of administration are preferred.

The compounds of the present disclosure can be used alone or synergistically with other drugs or carriers applied to tumor therapy.

When the compounds of the present disclosure are used in combination with other therapeutic drugs, suitable doses of the compounds of the present disclosure, as well as the drug carrier or other drug, are usually made into appropriate pharmaceutical forms. Thus, the present disclosure also provides pharmaceutical synergistic drug forms which comprise an effective dose of the compounds described herein as well as at least one other type of pharmaceutically usable carrier or therapeutic drug.

In yet another object, the present disclosure also provides use of the compounds described in any of the preceding paragraphs of the present disclosure (including the compounds shown in formula (I), formula (V-1), (V-2), (V-3), (V-4), and formula (VI)), or isomers, or pharmaceutically acceptable salts or deuterium substituents thereof, or pharmaceutical compositions described therein, in the preparation of a medicament for treating tumors.

According to some specific embodiments of the present disclosure, wherein said medicament for treating tumors is a medicament that is used in conjunction with radiation therapy for tumors.

In yet another object, the present disclosure also provides a method of treating a tumor, said method comprising administering a compound described in any of the preceding embodiments of the present disclosure (including compounds shown in formula (I), formula (V-1), (V-2), (V-3), (V-4), and formula (VI)), or isomers, or pharmaceutically acceptable salts or deuterated substituents thereof, or said pharmaceutical compositions.

According to some specific embodiments of the present disclosure, wherein said method comprises administering a compound or isomer thereof or a pharmaceutically acceptable salt or deuterium substitute thereof, or said pharmaceutical composition, at the time of radiation therapy to a tumor.

According to some specific embodiments of the present disclosure, wherein said radiation for radiation therapy is selected from alpha rays, beta rays, gamma rays or X-rays.

It is to be understood that “at the time of radiation therapy to a tumor” as described herein may include an acceptable period during the administration of the radiation, as well as prior to the administration of the radiation.

The compounds of the present disclosure, or isomers, or pharmaceutically acceptable salts or deuterates thereof, or pharmaceutical compositions as described, can be used in the treatment of various tumors. They are particularly suitable for the preparation of therapeutic drugs for the treatment of cancers for which radiation therapy is clinically the first line of treatment. The compounds of the present disclosure, in particular the compounds of formula (I), can not only be used in combination with radiation therapy for enhancing the efficacy of the radiation therapy, thereby reducing the dose and frequency of administration of the radiation therapy, but are also expected to overcome problems such as resistance to radiation therapy. Meanwhile, these radiotherapy-responsive PROTAC molecules exist in the form of prodrugs in the absence of irradiation because key active sites, especially on the target protein-binding ligand and E3 ligase ligand, are modified with “chemical cages”. These prodrugs do not function in protein degradation, thus reducing the overall toxicity of the drug. However, after local irradiation of a specific lesion, such as a tumor site, the active substance produced by the irradiation induces the irradiation-responsive “chemical cage” to undergo a specific chemical reaction, releasing compounds with protein-degrading activity. These released compounds efficiently degrade the target proteins at the site of the lesion, thereby enhancing the effect of radiation therapy.

It is to be understood that “optionally” as described in the present disclosure, it is to be understood that the event or situation may or may not occur. For example, the said “optionally, said alkyl group is further substituted with H, halogen, hydroxyl, carboxyl, amino, cyano, carbonyl, or alkyl group of C1-5” should be understood as the said alkyl group may or may not be substituted with a substituent group of H, halogen, hydroxyl, carboxyl, etc., as described above. The substituents may or may not be substituted with the aforementioned H, halogen, hydroxyl, carboxyl, etc.

The “isomers” referred to in the present disclosure include geometrical isomers (e.g., chiral isomers, cis-trans isomers, etc.) and reciprocal isomers.

### BRIEF DESCRIPTION OF THE DRAWINGS

FIG. 1. Fluorescence intensity changes of the compound after irradiation in Experimental Example 2.

FIG. 2. HPLC analysis of the compound after irradiation in Experimental Example 3.

FIG. 3. Degradation effect of PROTAC compound AS2 on BRD4.

FIG. 4. Degradation effect of radiation-responsive PROTAC compound A2 on BRD4 before irradiation.

FIG. 5. Degradation effect of radiation-responsive PROTAC compound A2 on BRD4 when co-treated with X-ray irradiation.

FIG. 6. Apoptosis detection by Annexin V/PI double staining.

FIG. 7. Timeline for in vivo antitumor activity study of AS2 and A1 molecules.

FIG. 8. Tumor growth curves after combined treatment with AS2 and radiotherapy.

FIG. 9. Tumor weights after combined treatment with AS2 and radiotherapy.

FIG. 10. Tumor growth curves after combined treatment with A1 and radiotherapy.

FIG. 11. Tumor weights after combined treatment with A1 and radiotherapy.

### DETAILED DESCRIPTION

The technical solutions of the present disclosure are described in detail below with reference to the accompanying drawings and embodiments, but the scope of protection of the present invention includes but is not limited thereto.

### Example 1: Preparation of X-Ray-Responsive Proteolysis-Targeting Chimera Prodrugs and Analogs Thereof in the Present Disclosure

Based on the above technical solution, the disclosure firstly provides a method for the preparation of compounds A1 and its analogs B1, C1, D1, comprising the following steps:

### a) First, an X-Ray Responsive Functional Unit, S1, was Synthesized.

A 100 mL round bottom flask was charged with a mixture of acetone/H₂O (30 mL, 2:1). 2,3,4,5,6-pentafluorobenzaldehyde (1.53 g, 1.0 mL, 7.77 mmol, 1.00 eq.) and NaN₃ (0.532 g, 8.18 mmol, 1.06 eq) were added and the mixture was refluxed at 80° C. overnight. The reaction mixture was cooled to room temperature, and acetone was removed. H₂O (30 mL) was added, and then the mixture was extracted with Et₂O (3×50 mL). The organic phases were washed with brine (50 mL), dried over MgSO₄, filtered and the solvent was evaporated. The residue was purified by flash column chromatography (PE/AcOEt=40:1) as a pale-yellow solid.

¹H NMR (CDCl₃, 400 MHz) δ: 10.26 (s, 1H).

¹³C NMR (CDCl₃, 100 MHz) δ: 181.60, 148.35, 145.81, 141.57, 138.86, 126.34, 110.67.

¹⁹F NMR (CDCl₃, 376 MHz) δ: −144.92, −150.97.

The solid (1.00 g, 4.56 mmol, 1.0 eq) was dissolved in acetic acid (15 mL), and Me₂NH·BH₃ (0.322 g, 5.47 mmol, 1.2 eq) was added. The reaction was stirred at room temperature for 2 h. H₂O (50 mL) was added, then the mixture was extracted with Et₂O (3×30 mL). The organic phases were washed with 15% Na₂CO₃ (2×50 mL) and brine (50 mL), dried over MgSO₄, filtered and the solvent was evaporated. The residue was purified by flash column chromatography (PE/AcOEt=5:1) as a pale-yellow solid (856.4 mg, 85%). The solid obtained is compound S1.

¹H NMR (DMSO-d₆, 400 MHz) δ: 4.53 (s, 1H).

¹³C NMR (DMSO-d₆, 100 MHz) δ: 176.39, 146.30, 143.98, 141.85, 139.14, 115.78, 51.13.

¹⁹F NMR (DMSO-d₆, 470 MHz) δ: −145.39, −152.90.

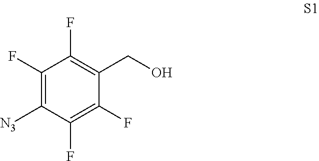 [SMILES: [N-]=[N+]=Nc1c(F)c(F)c(CO)c(F)c1F]

### b) Synthesis of S2, a Ligand for Target Protein of Interest

Tert-butyl(S)-2-(4-(4-chlorophenyl)-2,3,9-trimethyl-6H-thieno[3,2-f][1,2,4]triazolo[4, 3-a][1,4]diazepin-6-yl)acetate (1.0 g, 2.2 mmol) was dissolved in CH₂Cl₂ (20 mL) and CF₃COOH (3 mL) was slowly added in the solution. The mixture was stirred at room temperature for 12 h. After TLC analysis indicated that the reaction was complete, the solvent was evaporated in vacuo to afford the crude product, which was purified by silica gel column chromatography (CH₂Cl₂:MeOH=100:6) to give compound S2 (771.8 mg, 88% yield) as a white solid.

¹H NMR (CDCl₃, 300 MHz) δ: 1.69 (s, 3H), 2.41 (s, 3H), 2.70 (s, 3H), 3.56-3.76 (m, 2H), 4.64 (t, J=6.9 Hz, 2H), 7.35 (d, J=8.25 Hz, 2H), 7.44 (d, J=8.22 Hz, 2H).

¹³C NMR (CDCl₃, 75 MHz) δ: 173.74, 164.20, 155.29, 150.02, 137.00, 136.30, 131.87, 131.29, 131.03, 130.62, 129.96, 128.76, 77.46, 77.04, 76.61, 53.70, 36.73, 14.43, 13.14, 11.69.

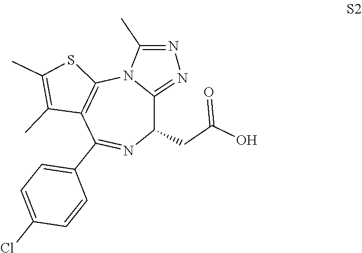 [SMILES: Cc1sc2c(c1C)C(c1ccc(Cl)cc1)=N[C@@H](CC(=O)O)c1nnc(C)n1-2]

### c) Synthesis of Compound AS1

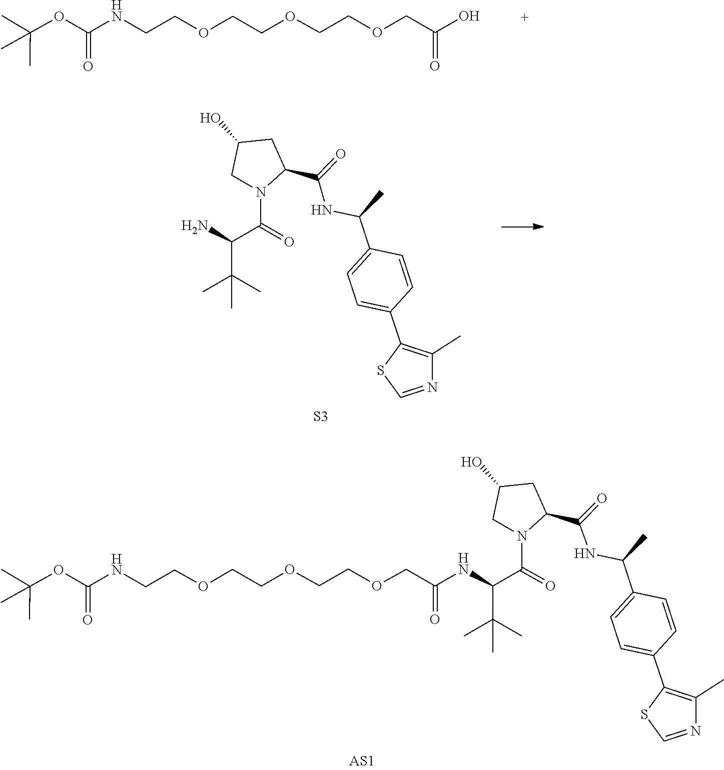 [SMILES: CC(C)(C)OC(=O)NCCOCCOCCOCC(=O)O ; Cc1ncsc1-c1ccc([C@H](C)NC(=O)[C@@H]2C[C@@H](O)CN2C(=O)[C@H](N)C(C)(C)C)cc1 ; "S3" ; Cc1ncsc1-c1ccc([C@H](C)NC(=O)[C@@H]2C[C@@H](O)CN2C(=O)[C@H](NC(=O)COCCOCCOCCNC(=O)OC(C)(C)C)C(C)(C)C)cc1]

HATU (2.5 g, 6.6 mmol), DIPEA (0.85 g, 6.6 mmol) and 5,8,11-trioxa-2-azatridecanedioic acid 1-tert-butyl ester (1.0 g, 3.3 mmol) were dissolved in DMF (12 mL). E3 ligase ligand S3 (1.47 g, 3.3 mmol) was slowly added into the solution. The reaction mixture was stirred for 24 h at room temperature. After the reaction was completed, the mixture was diluted with EtOAc (100 mL×3), washed with water (50 mL) and saturated NaCl solution (50 mL). The organic layer was dried over anhydrous Na₂SO₄, filtered, concentrated under reduced pressure and purified by silica gel column chromatography (CH₂Cl₂:MeOH=100:5) to give compound AS1 (1.67 g, 69% yield) as an oily liquid.

¹H NMR (CDCl₃, 300 MHz) δ: 1.69 (s, 3H), 2.41 (s, 3H), 2.70 (s, 3H), 3.56-3.76 (m, 2H), 4.64 (t, J=6.9 Hz, 2H), 7.35 (d, J=8.25 Hz, 2H), 7.44 (d, J=8.22 Hz, 2H).

¹³C NMR (CDCl₃, 75 MHz) δ: 173.74, 164.20, 155.29, 150.02, 137.00, 136.30, 131.87, 131.29, 131.03, 130.62, 129.96, 128.76, 77.46, 77.04, 76.61, 53.70, 36.73, 14.43, 13.14, 11.69.

Similarly, 5,8,11-trioxa-2-azatridecanedioic acid-1-tert-butyl ester was replaced with 2,2-dimethyl-4-oxo-3,8,12-trioxa-5-azatetradecane-14-oleic acid, or 11-((tert-butoxycarbonyl)amino)undecanoic acid in the above reaction to give compounds BS1, CS1, respectively, with the molecular structures shown in Table 1:

TABLE 1

| | | ESI molecular weight: |
|---|---|---|
| Structure | No. | [M + H]⁺ |
| 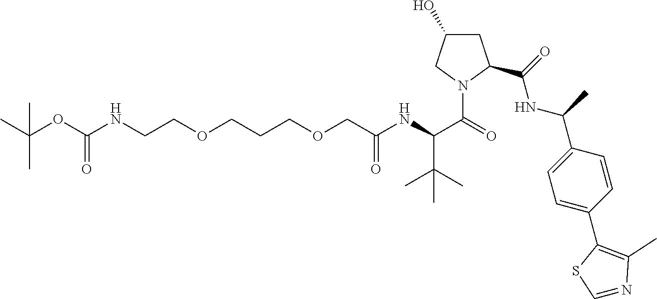 [SMILES: Cc1ncsc1-c1ccc([C@H](C)NC(=O)[C@@H]2C[C@@H](O)CN2C(=O)[C@H](NC(=O)COCCCOCCNC(=O)OC(C)(C)C)C(C)(C)C)cc1] | BS1 | 704.9 |
| 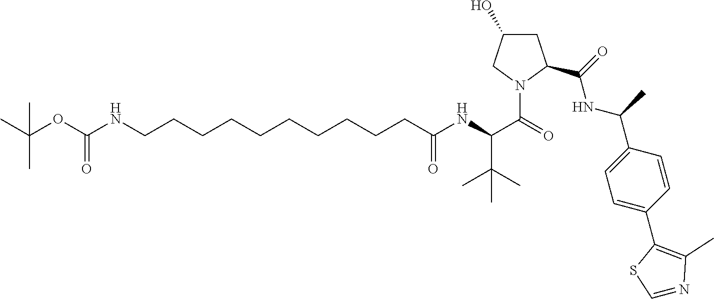 [SMILES: Cc1ncsc1-c1ccc([C@H](C)NC(=O)[C@@H]2C[C@@H](O)CN2C(=O)[C@H](NC(=O)CCCCCCCCCCNC(=O)OC(C)(C)C)C(C)(C)C)cc1] | CS1 | 729.0 |

### d) Synthesis of Compound AS2

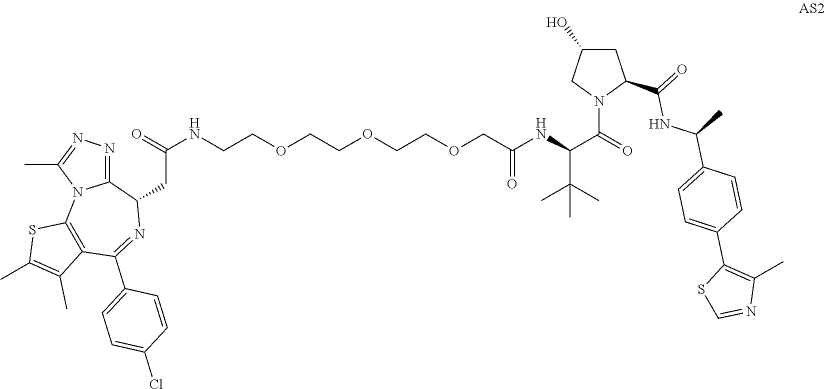

Compound AS1 (1.0 g, 1.4 mmol) was slowly added into a mixed solution of CF₃COOH (3 mL) and CH₂Cl₂ (15 mL). The mixture was stirred for 6 h at room temperature. The solvent was evaporated under reduced pressure. Subsequently, the product obtained above, compound S2 (840 mg, 2.1 mmol), HATU (799 mg, 2.1 mmol) and DIPEA (271 mg, 2.1 mmol) was slowly added in DMF (20 mL), and the mixture was stirred for 24 h at room temperature. After the reaction was completed, the reaction mixture was diluted with EtOAc (50 mL×3), washed with water (100 mL) and saturated NaCl solution (50 mL×3). The organic layer was dried over anhydrous Na₂SO₄, filtered, concentrated in vacuo and purified by silica gel column chromatography (CH₂Cl₂:MeOH=100:3) to give compound AS2.

¹H NMR (DMSO-d₆, 400 MHz) δ: 0.95 (s, 3H), 1.37 (s, 3H), 1.76-1.82 (m, 1H), 2.03-2.07 (m, 1H), 2.46 (s, 3H), 3.08 (d, J=5.68, 2H), 3.40 (t, J=5.80, 2H), 3.52-3.60 (m, 10H), 3.96 (s, 2H), 4.29 (s, 1H), 4.45 (t, J=15.96, 1H), 4.56 (d, J=9.52, 1H), 4.91 (t, J=6.96, 1H), 5.11 (d, J=3.32, 1H), 6.72 (s, 1H), 7.36-7.38 (m, 3H), 7.42-7.44 (m, 2H), 8.42 (d, J=7.56, 1H), 8.98 (s, 1H).

¹³C NMR (DMSO-d₆, 100 MHz) δ: 170.93, 169.50, 168.99, 156.05, 151.91, 148.23, 145.16, 131.58, 130.18, 129.30, 126.81, 78.04, 70.93, 70.29, 70.09, 69.99, 69.67, 69.25, 59.04, 56.99, 56.18, 55.36, 48.22, 38.19, 36.20, 28.70, 26.71, 22.91, 16.45.

Similarly, AS1 was replaced with BS1 or CS1 in the above reaction to obtain compounds BS2, CS2, respectively, with the molecular structures shown in Table 2.

TABLE 2

| | | ESI |
|---|---|---|
| | | molecular |
| | | weight: |
| Structure | No. | [M + H]⁺ |
| 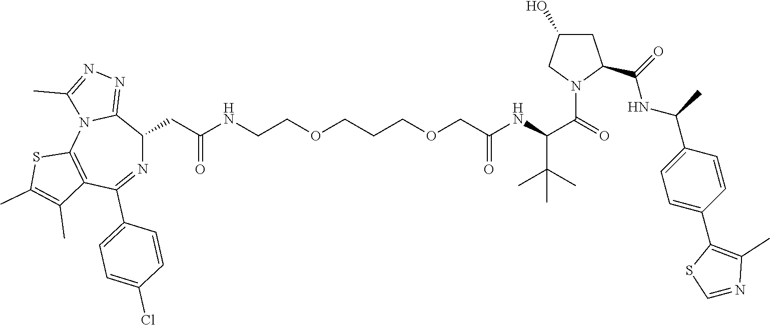 | BS2 | 987.7 |
| 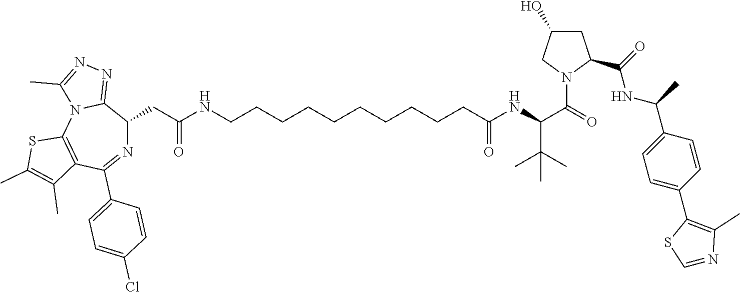 | CS2 | 1011.7 |

### d) Synthesis of Compound DS1

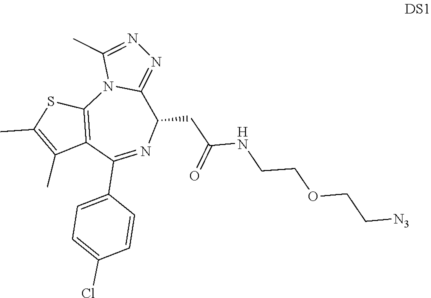 [SMILES: Cc1sc2c(c1C)C(c1ccc(Cl)cc1)=N[C@@H](CC(=O)NCCOCCN=[N+]=[N-])c1nnc(C)n1-2]

HATU (2.5 g, 6.6 mmol), DIPEA (0.85 g, 6.6 mmol) and S2 (1.0 g, 3.3 mmol) were dissolved into DMF (12 mL). 2-(2-azidoethoxy)ethan-1-amine (429.5 mg, 3.3 mmol) was slowly added to the solution. Subsequently, the mixture was stirred and reacted at room temperature for 24 hours. After completion of the reaction, the mixture was diluted with EtOAc (100 mL×3) and washed with water (50 mL) and saturated NaCl solution (50 mL). The organic layer was dried with anhydrous Na₂SO₄, filtered, concentrated under reduced pressure and purified by silica gel column chromatography (PE:EA=1:1) to give compound DS1 (1.02 g, 61% yield) as a white solid.

¹H NMR (400 MHz, DMSO-d6) δ 8.29 (t, J=5.7 Hz, 1H), 7.49 (d, J=8.4 Hz, 2H), 7.42 (d, J=8.4 Hz, 2H), 4.51 (dd, J=7.9, 6.1 Hz, 1H), 3.62 (t, J=4.9 Hz, 2H), 3.50 (t, J=5.9 Hz, 2H), 3.41 (t, J=4.9 Hz, 2H), 3.35-3.16 (m, 4H), 2.59 (s, 3H), 2.41 (s, 3H), 1.62 (s, 3H).

¹³C NMR (101 MHz, DMSO-d6) δ 169.69, 162.98, 155.09, 149.79, 136.76, 135.20, 132.26, 130.68, 130.13, 129.81, 129.55, 128.44, 69.00, 68.97, 53.82, 49.95, 38.53, 37.52, 14.03, 12.67, 11.28.

### f) Synthesis of Compound DS2

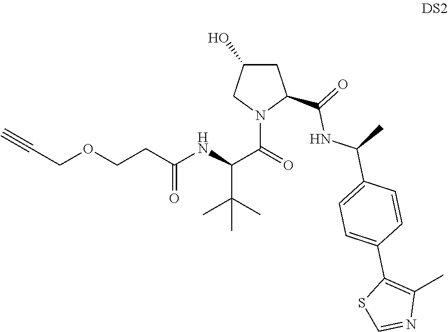 [SMILES: C#CCOCCC(=O)N[C@@H](C(=O)N1C[C@H](O)C[C@H]1C(=O)N[C@@H](C)c1ccc(-c2scnc2C)cc1)C(C)(C)C]

HATU (2.5 g, 6.6 mmol), DIPEA (0.85 g, 6.6 mmol), and 3-(prop-2-yn-1-yloxy)propionic acid (422.8 mg, 3.3 mmol) were dissolved into DMF (12 mL). E3 ligase ligand S3 (1.47 g, 3.3 mmol) was slowly added to the solution. Subsequently, the mixture was stirred and reacted at room temperature for 24 hours. After completion of the reaction, the mixture was diluted with EtOAc (100 mL×3) and washed with water (50 mL) and saturated NaCl solution (50 mL). The organic layer was dried with anhydrous Na₂SO₄, filtered, concentrated under reduced pressure and purified by silica gel column chromatography (CH₂Cl₂:MeOH=100:5) to give an oily liquid as compound DS2 (1.32 g, 72% yield).

¹H NMR (400 MHz, Methanol-d4) δ 8.88 (s, 1H), 7.51-7.38 (m, 4H), 4.65 (s, 1H), 4.60-4.48 (m, 4H), 4.36 (d, J=15.5 Hz, 1H), 4.16 (dd, J=2.4, 0.8 Hz, 2H), 3.90 (dt, J=11.1, 1.7 Hz, 1H), 3.84-3.72 (m, 3H), 2.85 (t, J=2.4 Hz, 1H), 2.60 (ddd, J=15.1, 7.5, 5.4 Hz, 1H), 2.53-2.44 (m, 4H), 2.22 (ddt, J=13.1, 7.7, 2.0 Hz, 1H), 2.08 (ddd, J=13.3, 9.1, 4.6 Hz, 1H), 1.04 (s, 9H).

¹³C NMR (101 MHz, Methanol-d4) δ 174.46, 173.49, 172.20, 152.84, 149.05, 140.28, 133.35, 131.53, 130.38, 128.98, 80.42, 76.05, 71.09, 66.92, 60.82, 59.03, 58.91, 57.99, 43.71, 38.91, 37.12, 36.72, 27.02, 15.79.

### g) Synthesis of Compound DS3

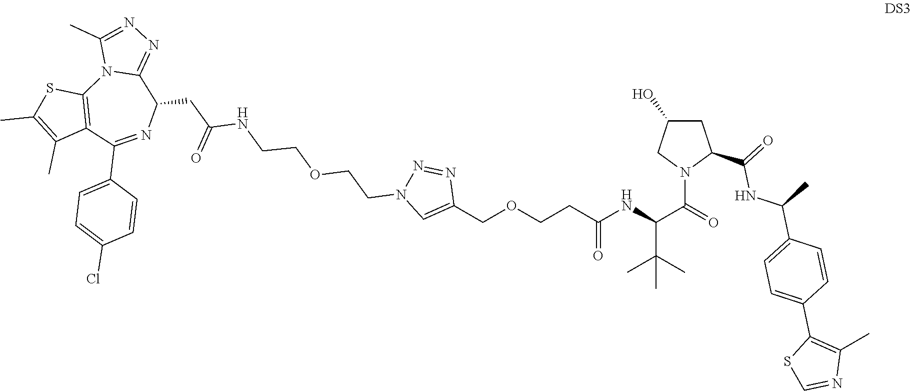

DS1 (50 mM DMSO solution, 2.3 mL, 0.12 mmol) and DS2 (50 mM DMSO solution, 2.3 mL, 0.12 mmol) were mixed and diluted with 4 mL of tert-butanol, and then pre-mixed tris(3-hydroxypropyltriazolylmethyl)amine (THPTA, 50 mM aqueous solution, 4.7 mL, 0.23 mmol) and copper sulfate (II) (100 mM aqueous solution, 1.2 mL, 0.12 mmol) solution were added to the above solution. Finally, freshly prepared sodium L-ascorbate solution (200 mM in water, 3 mL, 0.59 mmol) was added to it and the reaction mixture was stirred at room temperature overnight. The solution was diluted with 50 mL of water and extracted twice with 50 mL each of dichloromethane and ethyl acetate. The organic layers were dried with anhydrous sodium sulfate, combined, concentrated under reduced pressure and passed through silica gel column chromatography (CH₂Cl₂:MeOH=100:10) to give DS3 as a white solid (101.0 mg, 81% yield).

¹H NMR (400 MHz, Chloroform-d) δ 8.71 (s, 1H), 7.98 (s, 1H), 7.63-7.55 (m, 2H), 7.46-7.29 (m, 9H), 4.76 (t, J=8.2 Hz, 1H), 4.70-4.47 (m, 7H), 4.46-4.41 (m, 1H), 4.35 (dd, J=15.1, 5.4 Hz, 1H), 4.15 (d, J=11.4 Hz, 1H), 3.89-3.71 (m, 3H), 3.70-3.63 (m, 1H), 3.59 (dd, J=11.3, 3.3 Hz, 1H), 3.54-3.22 (m, 6H), 2.65 (s, 3H), 2.59-2.47 (m, 4H), 2.46-2.32 (m, 4H), 2.19 (dd, J=13.4, 7.9 Hz, 1H), 1.65 (s, 3H), 0.96 (s, 9H).

### h) Synthesis of Compound AS3

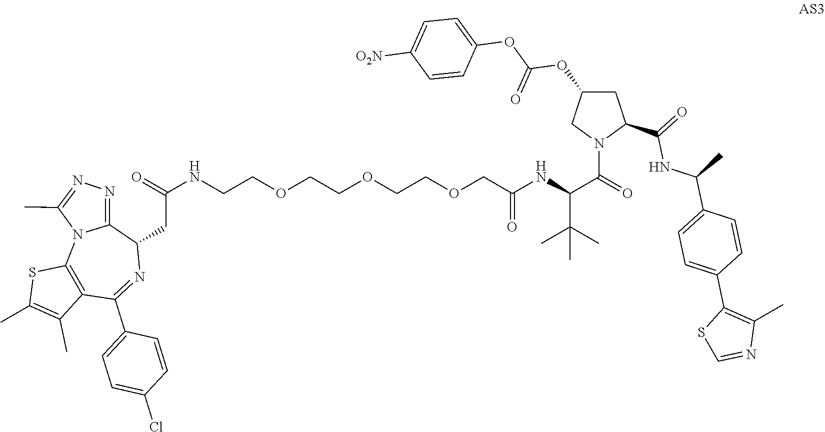

Compound AS2 (200 mg, 0.2 mmol), 4-nitrobenzoyl chloride (201 mg, 1.0 mmol) and DMAP (24 mg, 0.2 mmol) were slowly added to anhydrous CH₂Cl₂ (8.0 mL) and the mixture was stirred for 12 h at room temperature. After completion of the reaction, the solvent was removed to give the crude product, which was subsequently purified by silica gel column chromatography (CH₂Cl₂:MeOH=100:3) to give compound S5 (116 mg, 49% yield) as a white solid.

¹H NMR (DMSO-d₆, 400 MHz) δ: 1.00 (s, 9H), 1.41 (d, J=6.96, 3H), 1.62 (s, 3H), 2.07-2.14 (m, 1H), 2.40 (s, 3H), 2.46-2.48 (m, 4H), 2.60 (s, 3H), 3.25-3.31 (m, 4H), 3.49 (t, J=5.96, 2H), 3.57-3.66 (m, 8H), 3.85 (dd, J=12.08, 3.12, 1H), 4.01 (d, J=1.36, 2H), 4.33 (d, J=8.16, 1H), 4.47 (d, J=8.84, 1H), 4.52-4.58 (m, 2H), 4.92-4.99 (m, 1H), 5.31 (s, 1H), 7.39-7.52 (m, 9H), 7.64-7.66 (m, 2H), 8.27-8.34 (m, 3H), 8.51 (d, J=7.64, 1H), 8.99 (s, 1H).

¹³C NMR (DMSO-d₆, 100 MHz) δ: 170.16, 170.09, 169.86, 169.83, 163.47, 155.70, 155.60, 151.92, 150.26, 148.24, 145.71, 144.98, 137.23, 135.71, 132.73, 131.57, 131.16, 130.60, 130.31, 130.26, 130.05, 129.34, 128.90, 126.83, 125.79, 123.24, 79.65, 79.10, 70.87, 70.28, 70.11, 70.07, 69.95, 69.71, 58.49, 57.01, 54.32, 53.87, 48.38, 39.11, 38.01, 35.18, 26.65, 22.89, 16.44, 14.49, 13.12, 11.75.

Similarly, AS2 in the above reaction was replaced with BS2, CS2 or DS3 to obtain compounds BS3, CS3, DS4, respectively, with the molecular structures shown in Table 3.

TABLE 3

| | | ESI |
|---|---|---|
| | | molecular |
| | | weight: |
| Structure | No. | [M + H]⁺ |
| 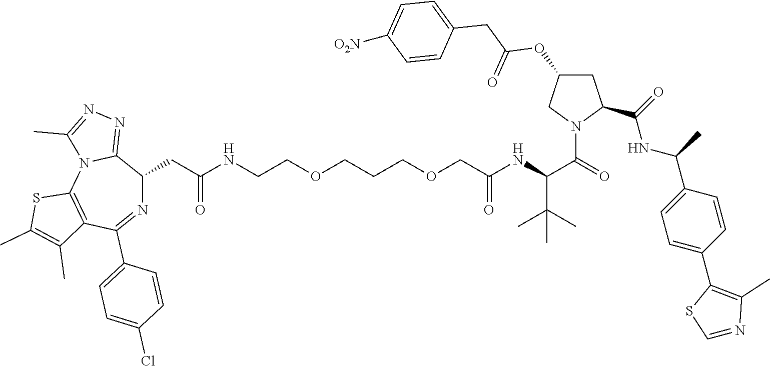 | BS3 | 1152.8 |
| 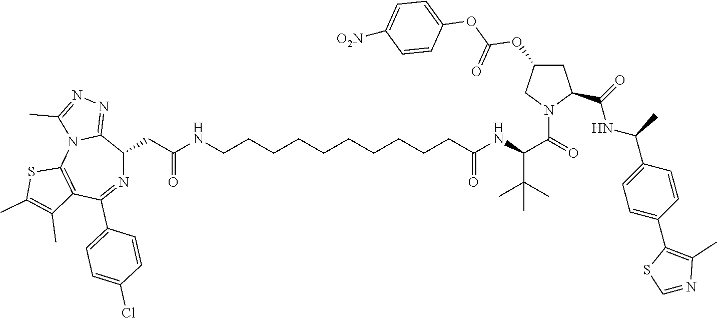 | CS3 | 1176.9 |
| 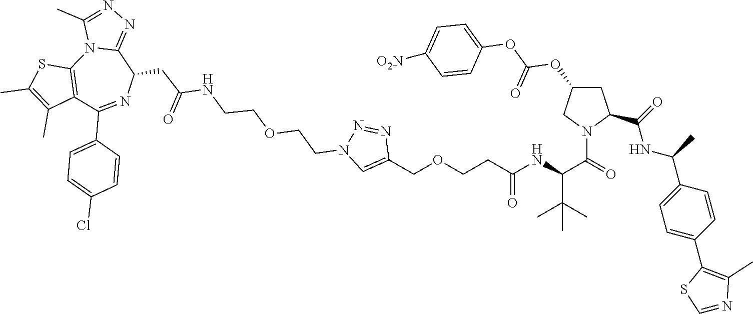 | DS4 | 1232.5 |

### i) Synthesis of Compound A1

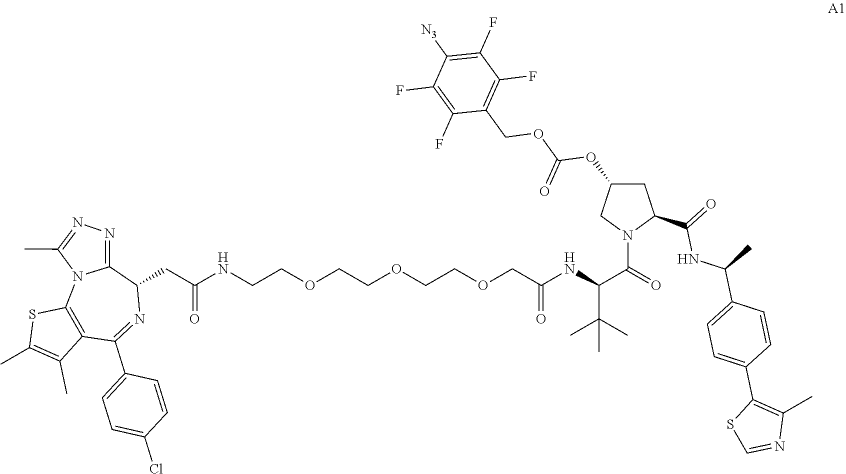

Compound AS3 (100 mg, 0.08 mmol), compound S1 (66 mg, 0.3 mmol) and DMAP (37 mg, 0.3 mmol) were slowly dissolved in anhydrous DMF (5.0 mL) and the mixture was stirred for 12 h at 50° C. The crude product was obtained by evaporating the solvent under reduced pressure and purified by silica gel column chromatography (CH₂Cl₂:MeOH=100:5) to give a white solid as compound A1 (56 mg, 55% yield).

¹H NMR (DMSO-d₆, 400 MHz) δ: 8.94 (s, 1H), 8.52 (d, J=7.44, 1H), 8.29 (t, J=4.84, 1H), 7.46-7.36 (m, 7H), 5.27-5.19 (m, 3H), 4.91-4.88 (m, 1H), 4.52-4.42 (m, 3H), 4.00-3.96 (m, 16H), 3.60-3.58 (m, 10H), 3.39-3.19 (m, 6H), 2.61 (s, 3H), 2.45 (s, 3H), 2.40 (s, 3H), 2.04-1.99 (m, 1H), 1.61 (s, 3H), 1.39 (d, J=6.80, 3H), 0.94 (s, 9H).

¹³C NMR (DMSO-d₆, 100 MHz) δ: 170.14, 170.05, 169.62, 169.34, 163.46, 155.60, 153.72, 151.94, 150.25, 148.25, 144.97, 137.25, 135.69, 132.74, 131.56, 131.16, 130.61, 130.30, 130.24, 130.05, 129.33, 128.91, 126.80, 109.37, 77.95, 70.91, 70.28, 70.07, 69.96, 69.71, 58.47, 57.02, 56.53, 54.32, 48.33, 39.11, 37.99, 35.58, 34.98, 26.61, 22.89, 16.44, 14.50, 13.14, 11.75.

¹⁹F NMR (DMSO-d₆, 470 MHz) δ: −143.03, −152.23.

HRMS (ESI) for C₅₈H₆₃ClF₄N₁₂O₁₀S₂[M+Na]⁺: calcd.: 1285.37539; found: 1285.37580.

Similarly, substitution of AS3 for BS3, CS3 or DS4 in the above reaction gave compounds B1, C1, D1, respectively, with the molecular structures shown in Table 4.

TABLE 4

| | | ESI |
|---|---|---|
| | | molec- |
| | | ular |
| | | weight: |
| | | [M + |
| Structure | No. | H]⁺ |
| 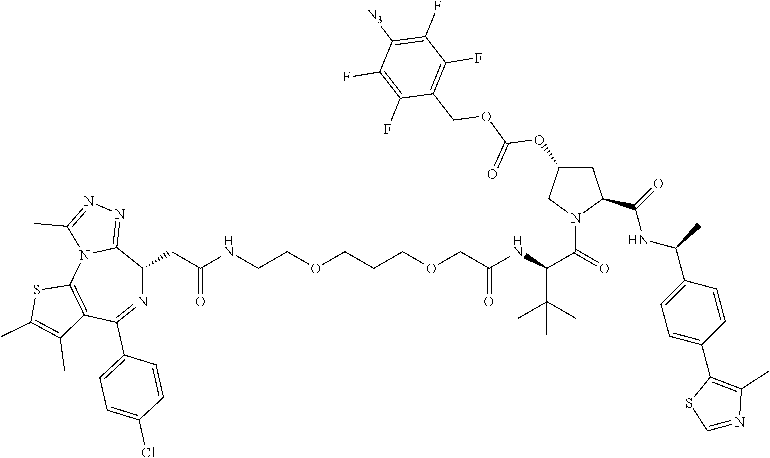 | B1 | 1234.8 |
| 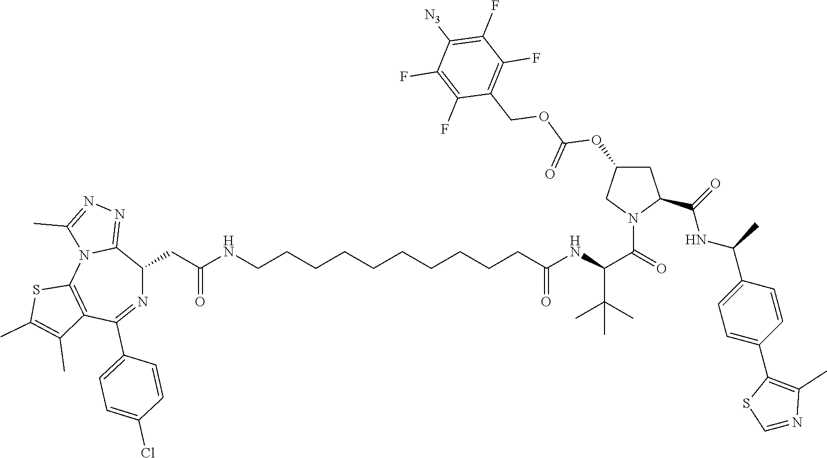 | C1 | 1258.9 |
| 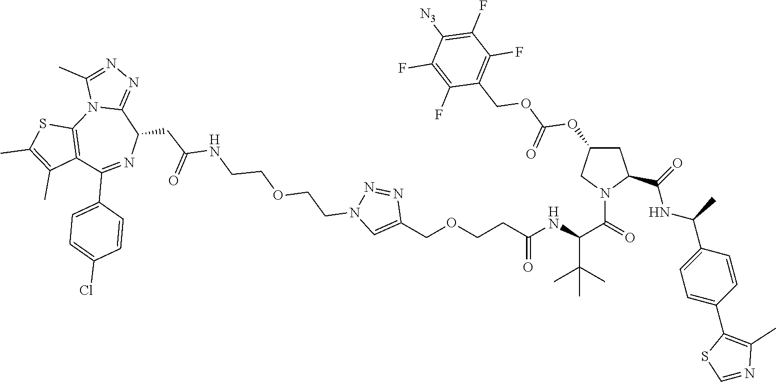 | D1 | 1314.5 |

### ii) Synthesis of Compounds A2 to D2

The synthetic operation of A2, B2, C2, and D2 is consistent with the operation of A1, B1, C1, and D1. Specifically, compound AS3 (or BS3, CS3, or DS4) as well as 3,5-dimethoxybenzyl alcohol and DMAP were slowly dissolved in anhydrous DMF and the mixture was stirred at 50° C. for 12 hours. The crude product was obtained by evaporating the solvent under reduced pressure and purified by silica gel column chromatography (CH₂Cl₂:MeOH=100:5) to give a white solid as compound A2 (56 mg, 55% yield) (or B2, C2 or D2).

TABLE 5

| | | ESI molecular |
|---|---|---|
| | | weight: |
| Structure | No. | [M + H]⁺ |
| 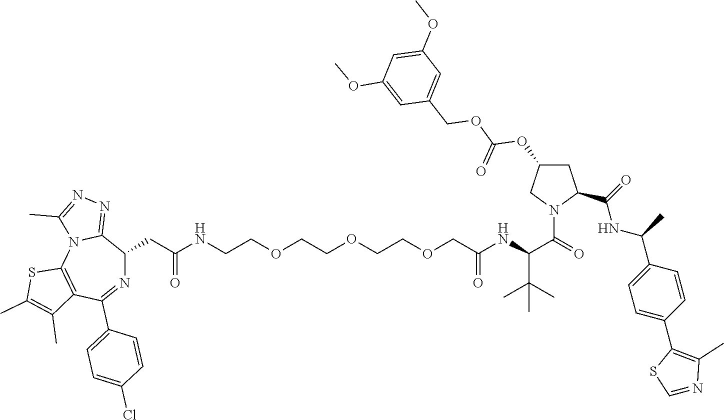 | A2 | 1209.8 |
| 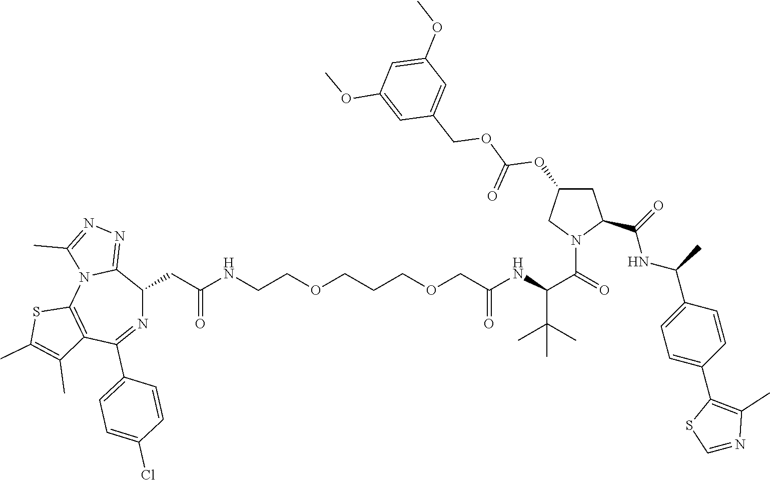 | B2 | 1179.9 |
| 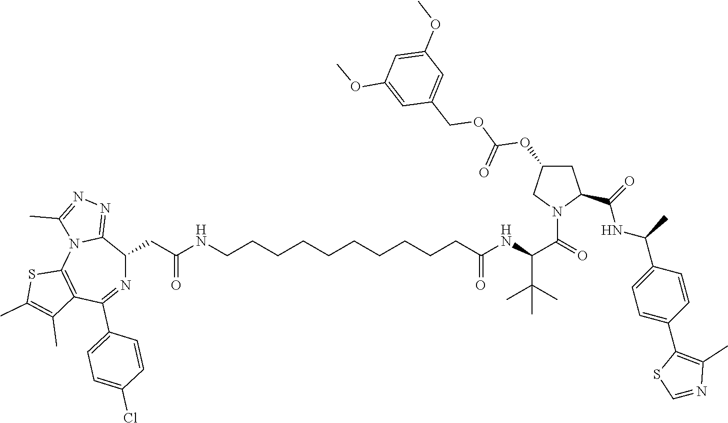 | C2 | 1204.1 |
| 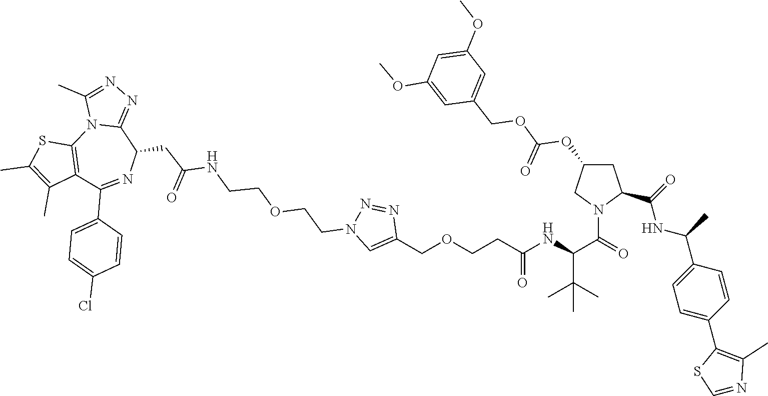 | D2 | 1260.3 |

### Synthesis of Compound ES2

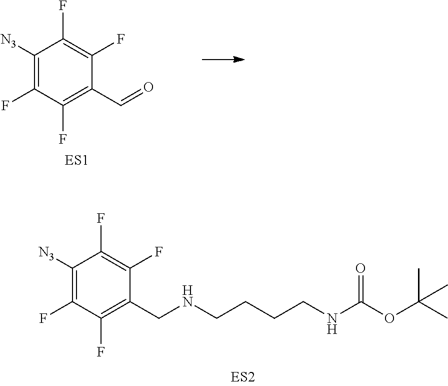 [SMILES: [N-]=[N+]=Nc1c(F)c(F)c(C=O)c(F)c1F ; CC(C)(C)OC(=O)NCCCCNCc1c(F)c(F)c(N=[N+]=[N-])c(F)c1F]

4-Azidotetrafluorobenzaldehyde (ES1, 5.5 g, 25.1 mmol), tert-butyl N-(5-aminopentyl)carbamate (4.7 g, 25.1 mmol), sodium triacetoxyborohydride (2.7 g, 12.6 mmol), and acetic acid (7.6 g, 126 mmol) were dissolved in anhydrous DCM and the reaction was heated up to 50° C. for 8 hours. After completion of the reaction, the mixture was diluted with EtOAc (100 mL 5×3) and washed with water (50 mL) and saturated NaCl solution (50 mL). The organic layer was dried with anhydrous Na₂SO₄, filtered, concentrated under reduced pressure and purified by silica gel column chromatography (CH₂Cl₂:EA=4:1) to give compound ES2 (6.5 g, 66% yield).

¹H NMR (DMSO-d₆, 300 MHz) δ: 1.36-1.39 (m, 2H), 1.42 (s, 12H), 1.49-1.52 (m, 2H), 2.53-2.57 (m, 2H), 3.17-3.20 (m, 2H), 3.76 (s, 2H), 4.16 (s, 1H), 6.76 (s, 1H).

¹³C NMR (DMSO-d₆, 75 MHz) δ: 155.9, 146.8, 135.6, 114.2, 103.8, 79.5, 49.2, 40.8, 35.8, 28.4, 27.3, 25.0.

Similarly, replacing 4-azidotetrafluorobenzaldehyde with 3,5-dimethoxybenzaldehyde in the above reaction yields compound ES3 with the molecular structure shown in Table 6.

TABLE 6

| | | ESI molecular weight: |
|---|---|---|
| Structure | No. | [M + H]⁺ |
| 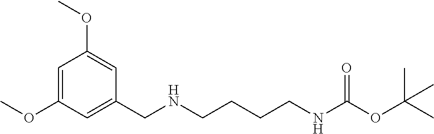 [SMILES: COc1cc(CNCCCCNC(=O)OC(C)(C)C)cc(OC)c1] | ES3 | 338.4 |
| ES3 | | |

### Synthesis of Compound S31

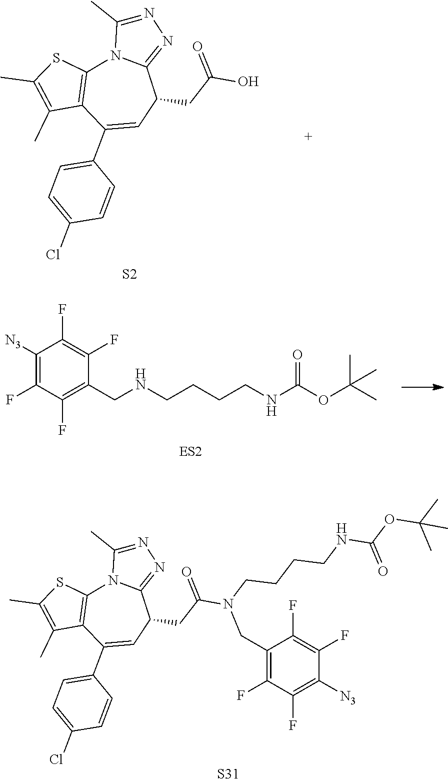

HATU (2.5 g, 6.6 mmol), DIPEA (0.85 g, 6.6 mmol) and S2 (1.0 g, 3.3 mmol) were dissolved into DMF (12 mL). ES2 (1.3 g, 3.3 mmol) was slowly added to the solution. Subsequently, the mixture was stirred and reacted at room temperature for 24 hours. After completion of the reaction, the mixture was diluted with EtOAc (100 mL 5×3) and washed with water (50 mL) and saturated NaCl solution (50 mL). The organic layer was dried with anhydrous Na₂SO₄, filtered, concentrated under reduced pressure and purified by silica gel column chromatography (CH₂Cl₂:MeOH=100:5) to afford compound S31 (3.7 g, 72% yield).

¹H NMR (DMSO-d₆, 300 MHz) δ: 1.42 (s, 12H), 1.49-1.52 (m, 4H), 2.14 (s, 3H), 2.35 (s, 3H), 2.36 (s, 3H), 2.71-2.96 (m, 2H), 3.18 (t, J=8.46 Hz, 2H), 4.72 (t, J=9.64 Hz, 1H), 4.91 (s, 1H), 6.76 (s, 1H), 7.51 (d, J=6.86 Hz, 2H), 7.75 (d, J=6.96 Hz, 2H).

¹³C NMR (DMSO-d₆, 75 MHz) δ: 172.0, 168.6, 155.9, 155.6, 149.3, 146.8, 137.1, 136.6, 136.0, 135.6, 133.9, 131.4, 130.6, 128.9, 120.8, 114.2, 103.8, 79.5, 55.4, 48.9, 39.9, 38.7, 35.8, 28.4, 27.3, 26.7, 25.0, 12.2, 10.4.

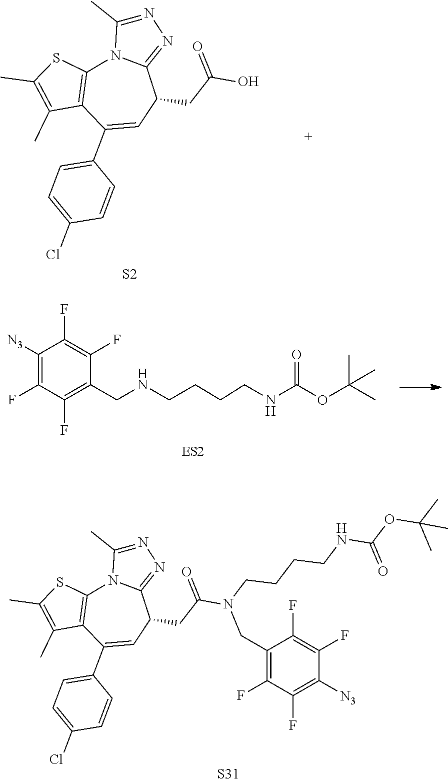

Similarly, replacing ES2 with ES3 in the above reaction gave compound S41 with the molecular structure shown in Table 7:

TABLE 7

| | | ESI molecular weight: |
|---|---|---|
| Structure | No. | [M + H]⁺ |
| 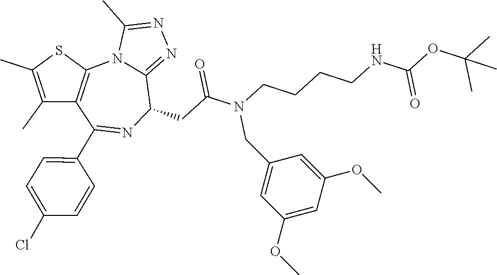 [SMILES: COc1cc(CN(CCCCNC(=O)OC(C)(C)C)C(=O)C[C@@H]2N=C(c3ccc(Cl)cc3)c3c(sc(C)c3C)-n3c(C)nnc32)cc(OC)c1] | S41 | 720.3 |
| S41 | | |

### Synthesis of Compound A3

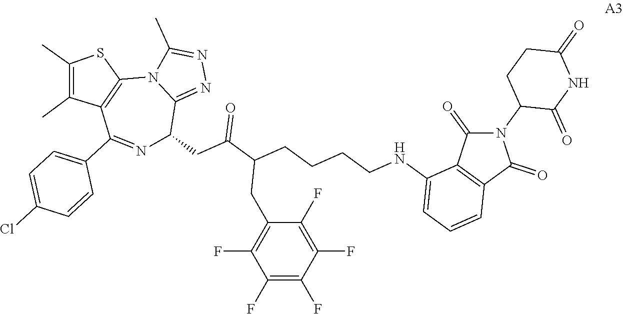 [SMILES: Cc1sc2c(c1C)C(c1ccc(Cl)cc1)=N[C@@H](CC(=O)C(CCCCNc1cccc3c1C(=O)N(C1CCC(=O)NC1=O)C3=O)Cc1c(F)c(F)c(F)c(F)c1F)c1nnc(C)n1-2]

Compound S31 (1.0 g, 1.4 mmol) was slowly added to a mixed solution of CH₂Cl₂ (15 mL) and CF₃COOH (3 mL) and the mixture was stirred at room temperature for 6 hours. The solvent in the mixture was subsequently evaporated under reduced pressure. The above products, DIPEA (724 mg, 5.6 mmol) and 2-(2,6-dioxaspiperidin-3-yl)-4-fluoroisoindoline-1,3-dione (387 mg, 1.4 mmol) were slowly added to a solution of DMF (20 mL) and the mixture was stirred for 24 h at 90° C. Upon completion of the reaction, the reaction mixture was diluted with EtOAc (50 mL×3) and washed with water (100 mL) and saturated NaCl solution (50 mL×3). The organic layer obtained after washing was dried with anhydrous Na₂SO₄, filtered, concentrated under reduced pressure and purified by silica gel column chromatography (CH₂Cl₂:MeOH=100:4) to give compound A3.

¹H NMR (DMSO-d₆, 300 MHz) δ: 1.49-1.52 (m, 4H), 2.02-2.11 (m, 2H), 1.49-1.52 (m, 4H), 2.14 (s, 3H), 2.21-2.27 (m, 2H), 2.35 (s, 3H), 2.36 (s, 3H), 2.71-2.96 (m, 2H), 3.18 (t, J=8.46 Hz, 2H), 3.29-3.31 (m, 2H), 4.44 (t, J=11.96 Hz, 1H), 4.72 (t, J=9.64 Hz, 1H), 4.91 (s, 1H), 6.79 (s, 1H), 6.98 (d, J=7.56 Hz, 1H), 7.25 (d, J=5.96 Hz, 1H), 7.51 (d, J=6.86 Hz, 2H), 7.59 (t, J=8.84 Hz, 1H) 7.75 (d, J=6.96 Hz, 2H).

¹³C NMR (DMSO-d₆, 75 MHz) δ: 173.8, 172.0, 168.9, 168.6, 167.9, 155.6, 149.3, 147.4, 146.8, 137.3, 137.1, 136.6, 136.0, 135.6, 133.9, 132.8, 131.4, 130.6, 128.9, 120.3, 117.0, 116.1, 114.2, 112.1, 103.8, 62.8, 55.4, 50.6, 48.9, 39.9, 38.7, 29.4, 27.0, 26.4, 21.3, 12.2, 10.4.

Similarly, replacing S31 with S41 in the above reaction gave compound B3 with the molecular structure shown in Table 8:

TABLE 8

| | | ESI molecular |
|---|---|---|
| Structure | No. | weight: [M + H]⁺ |
| 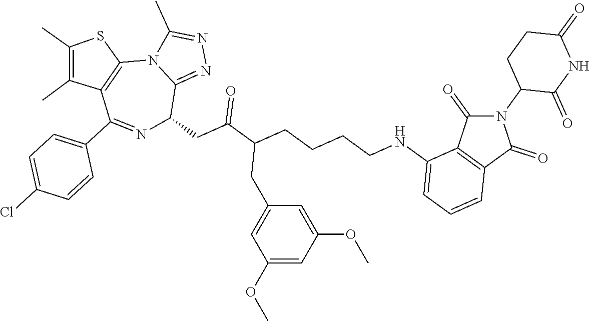 [SMILES: COc1cc(CC(CCCCNc2cccc3c2C(=O)N(C2CCC(=O)NC2=O)C3=O)C(=O)C[C@@H]2N=C(c3ccc(Cl)cc3)c3c(sc(C)c3C)-n3c(C)nnc32)cc(OC)c1] | B3 | 876.3 |

### Synthesis of Compound S51

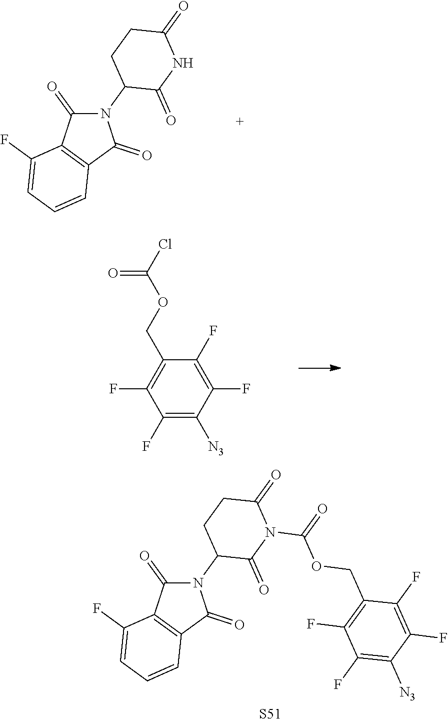 [SMILES: O=C1CCC(N2C(=O)c3cccc(F)c3C2=O)C(=O)N1 ; [N-]=[N+]=Nc1c(F)c(F)c(COC(=O)Cl)c(F)c1F ; [N-]=[N+]=Nc1c(F)c(F)c(COC(=O)N2C(=O)CCC(N3C(=O)c4cccc(F)c4C3=O)C2=O)c(F)c1F ; "S51"]

Compound 2-(2,6-dioxopiperidin-3-yl)-4-fluoroisoindoline-1,3-dione (1.0 g, 3.6 mmol) was slowly added to CH₂Cl₂ (15 mL) and cooled the solution to 0° C. Then NaH (103 mg, 4.3 mmol) was added and the reaction was carried out at 0° C. for 1 h. Subsequently, 4-azidotetrafluoromethylcarbonyl chloride (1.2 g, 4.3 mmol) and the mixture was stirred at room temperature for 6 hours. The solvent in the mixture was subsequently evaporated under reduced pressure. After completion of the reaction, the reaction mixture was diluted with EtOAc (50 mL×3) and washed with water (100 mL) and saturated NaCl solution (50 mL×3). The organic layer obtained after washing was dried with anhydrous Na₂SO₄, filtered, concentrated under reduced pressure and purified by silica gel column chromatography (CH₂Cl₂:PE=1:3) to give compound S51.

¹H NMR (DMSO-d₆, 300 MHz) δ: 2.02-2.27 (m, 4H), 2.02-2.11 (m, 2H), 4.44 (t, J=8.36 Hz, 1H), 5.05 (s, 2H), 7.61-7.63 (m, 2H), 7.74-7.75 (m, 1H).

¹³C NMR (DMSO-d₆, 75 MHz) δ: 174.2, 167.9, 158.0, 154.2, 147.2, 134.8, 133.8, 133.6, 119.3, 119.0, 117.9, 104.4, 60.0, 51.7, 26.6, 21.3.

Similarly, 4-azidotetrafluoromethylcarbonyl chloride was replaced with 3,5-dimethoxybenzylcarbonyl chloride in the above reaction to give compound S61 as in Table 9.

TABLE 9

| | | ESI molecular weight: |
|---|---|---|
| Structure | No. | [M + H]⁺ |
| 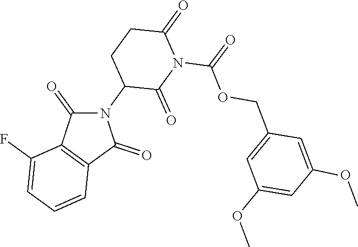 [SMILES: COc1cc(COC(=O)N2C(=O)CCC(N3C(=O)c4cccc(F)c4C3=O)C2=O)cc(OC)c1] | S61 | 470.1 |
| S61 | | |

### Synthesis of Compound ES7

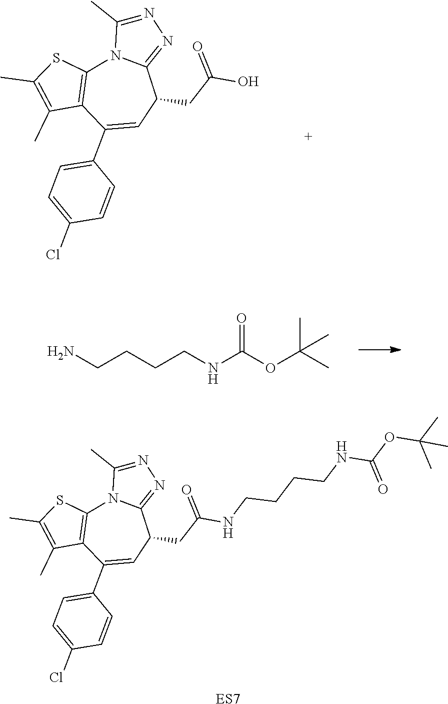 [SMILES: Cc1sc2c(c1C)C(c1ccc(Cl)cc1)=C[C@@H](CC(=O)O)c1nnc(C)n1-2 ; CC(C)(C)OC(=O)NCCCCN ; Cc1sc2c(c1C)C(c1ccc(Cl)cc1)=C[C@@H](CC(=O)NCCCCNC(=O)OC(C)(C)C)c1nnc(C)n1-2 ; "ES7"]

Compound S2 (1.0 g, 2.5 mmol), tert-butyl (4-aminobutyl)carbamate (470 mg, 2.5 mmol), HATU (1.14 g, 3.0 mmol), and DIPEA (388 mg, 3.0 mmol) were slowly added to a solution of DMF (20 mL) and the mixture was stirred for 24 hours at room temperature. After completion of the reaction, the reaction mixture was diluted with EtOAc (50 mL×3) and washed with water (100 mL) and saturated NaCl solution (50 mL×3). The organic layer obtained after washing was dried with anhydrous Na₂SO₄, filtered, concentrated under reduced pressure and purified by silica gel column chromatography (CH₂Cl₂:MeOH=100:3) to give compound ES7.

¹H NMR (DMSO-d₆, 300 MHz) δ: 1.42 (s, 12H), 1.51-1.53 (m, 4H), 2.14 (s, 3H), 2.35 (s, 3H), 2.36 (s, 3H), 2.71-3.10 (m, 4H), 3.18 (t, J=9.56 Hz, 2H), 4.72 (t, J=10.26 Hz, 1H), 6.76 (s, 2H), 7.51 (d, J=8.36 Hz, 2H), 7.75 (d, J=8.68 Hz, 1H).

¹³C NMR (DMSO-d₆, 75 MHz) δ: 173.3, 168.6, 155.9, 155.6, 149.3, 137.1, 136.6, 136.0, 133.9, 131.4, 130.6, 128.9, 120.3, 79.5, 55.1, 42.1, 38.9, 35.8, 28.4, 27.1, 27.0, 12.2, 10.5, 10.4.

### Synthesis of Compound C3

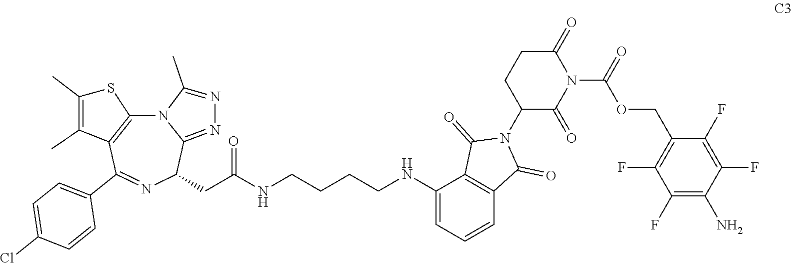 [SMILES: Cc1sc2c(c1C)C(c1ccc(Cl)cc1)=N[C@@H](CC(=O)NCCCCNc1cccc3c1C(=O)N(C1CCC(=O)N(C(=O)OCc4c(F)c(F)c(N)c(F)c4F)C1=O)C3=O)c1nnc(C)n1-2]

Compound ES7 (1.0 g, 1.8 mmol) was slowly added to a mixed solution of CH₂Cl₂ (15 mL) and CF₃COOH (3 mL) and the mixture was stirred at room temperature for 6 hours. The solvent in the mixture was subsequently evaporated under reduced pressure. The above products, compound S51 (941 mg, 1.8 mmol) and DIPEA (931 mg, 7.2 mmol) were slowly added to a solution of DMF (20 mL) and the mixture was stirred at room temperature for 24 hours. After completion of the reaction, the reaction mixture was diluted with EtOAc (50 mL×3) and washed with water (100 mL) and saturated NaCl solution (50 mL×3). The organic layer obtained after washing was dried with anhydrous Na₂SO₄, filtered, concentrated under reduced pressure and purified by silica gel column chromatography (CH₂Cl₂:MeOH=100:5) to give compound C3.

¹H NMR (DMSO-d₆, 300 MHz) δ: 1.49-1.52 (m, 4H), 2.02-2.11 (m, 2H), 2.14 (s, 3H), 2.21-2.27 (m, 2H), 2.35 (s, 3H), 2.36 (s, 3H), 2.71-3.03 (m, 4H), 3.30 (m, 2H), 4.44 (t, J=8.56 Hz, 1H), 4.72 (t, J=10.96 Hz, 1H), 5.05 (s, 2H), 6.79 (s, 2H), 6.98 (d, J=8.56 Hz, 1H), 7.25 (d, J=9.12 Hz, 1H), 7.51 (d, J=8.38 Hz, 2H), 7.59 (t, J=12.02 Hz, 1H), 7.75 (d, J=8.68 Hz, 1H).

¹³C NMR (DMSO-d₆, 75 MHz) δ: 174.7, 174.2, 173.3, 168.6, 167.9, 155.6, 154.2, 149.3, 147.4, 147.2, 137.3, 137.1, 136.6, 136.0, 134.8, 133.9, 132.8, 131.4, 130.6, 128.9, 120.3, 117.9, 117.0, 116.1, 112.1, 104.4, 60.0, 55.1, 51.7, 50.6, 42.1, 38.9, 27.4, 26.6, 26.1, 21.3, 12.2, 10.5, 10.4.

Similarly, S51 was replaced with S61 in the above reaction to obtain compound D3, as in Table 10.

TABLE 10

| | | ESI molecular |
|---|---|---|
| | | weight: |
| Structure | No. | [M + H]⁺ |
| 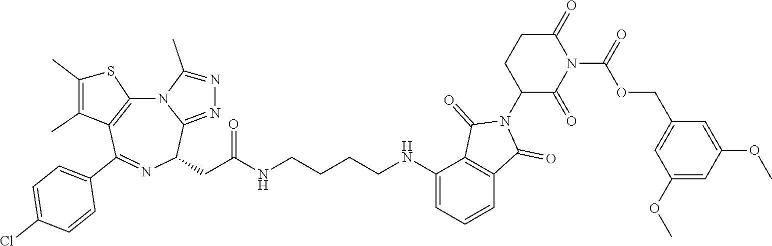 [SMILES: COc1cc(COC(=O)N2C(=O)CCC(N3C(=O)c4cccc(NCCCCNC(=O)C[C@@H]5N=C(c6ccc(Cl)cc6)c6c(sc(C)c6C)-n6c(C)nnc65)c4C3=O)C2=O)cc(OC)c1] | D3 | 920.3 |
| D3 | | |

### Synthesis of Compound M4

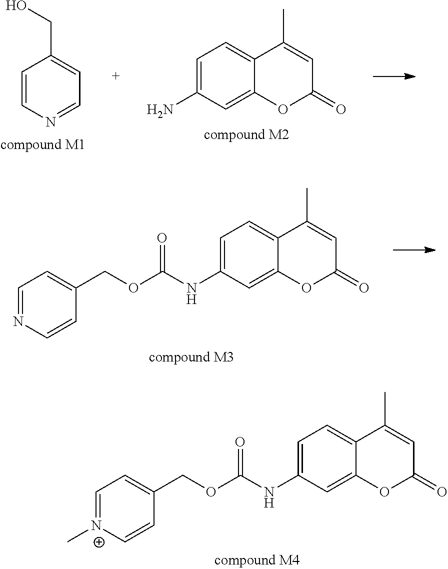 [SMILES: OCc1ccncc1 ; Cc1cc(=O)oc2cc(N)ccc12 ; Cc1cc(=O)oc2cc(NC(=O)OCc3ccncc3)ccc12 ; Cc1cc(=O)oc2cc(NC(=O)OCc3cc[n+](C)cc3)ccc12]

Dissolve compound M1 (1.75 g, 10 mmol, 1.0 eq.) and triphosgene (1.48 g, 5 mmol, 0.5 eq.) in DCM (35 mL, anhydrous), then add DIPEA (3.87 g, 30 mmol, 3.0 eq.). Under nitrogen protection, stir the reaction at room temperature for 30 minutes. Subsequently, add compound M2 (7-amino-4-methylcoumarin, 1.20 g, 11 mmol, 1.1 eq.) and continue the reaction at room temperature for 4 hours. After the reaction is complete, remove the solvent by rotary evaporation, then isolate the product by column chromatography. Dissolve compound M3 in a mixed solvent of methanol/iodomethane (5 mL), heat to 65° C. and react for 2 hours, then evaporate to dryness to obtain the crude product, which is recrystallized from methanol to yield a pink powder.

¹H NMR (400 MHz, DMSO) δ 2.40 (s, 3H), 4.33 (s, 3H), 5.53 (s, 2H), 6.28 (s, 1H), 7.44 (d, J=7.92 Hz, 1H), 7.57 (s, 1H), 7.75 (d, J=8.36 Hz, 1H), 8.10 (d, J=4.56 Hz, 2H), 8.98 (d, J=4.88 Hz, 2H), 10.61 (s, 1H).

### Synthesis of Compound N2

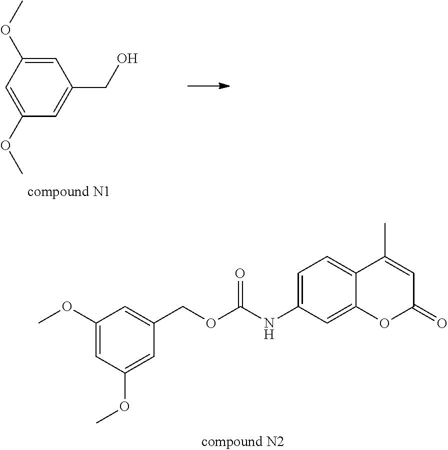 [SMILES: COc1cc(CO)cc(OC)c1 ; COc1cc(COC(=O)Nc2ccc3c(C)cc(=O)oc3c2)cc(OC)c1]

Dissolve compound N1 (2.55 g, 15.2 mmol, 1.0 eq.) and triphosgene (2.26 g, 7.6 mmol, 0.5 eq.) in DCM (35 mL, anhydrous), then add DIPEA (5.89 g, 45.6 mmol, 3.0 eq.). Under nitrogen protection, stir the reaction at room temperature for 30 minutes. Subsequently, add compound M2 (2.92 g, 16.7 mmol, 1.1 eq.) and continue the reaction at room temperature for 4 hours. After the reaction is complete, remove the solvent by rotary evaporation, then isolate the product by column chromatography.

¹H NMR (400 MHz, DMSO) δ, 2.39 (s, 3H), 3.75 (s, 6H), 5.12 (s, 2H), 6.25 (s, 1H), 6.48 (s, 1H), 6.61 (s, 2H), 7.43 (d, J=9.72 Hz, 1H) 7.56 (s, 1H), 7.72 (d, J=8.64 Hz, 1H), 10.31 (s, 1H).

### Synthesis of Compound O7

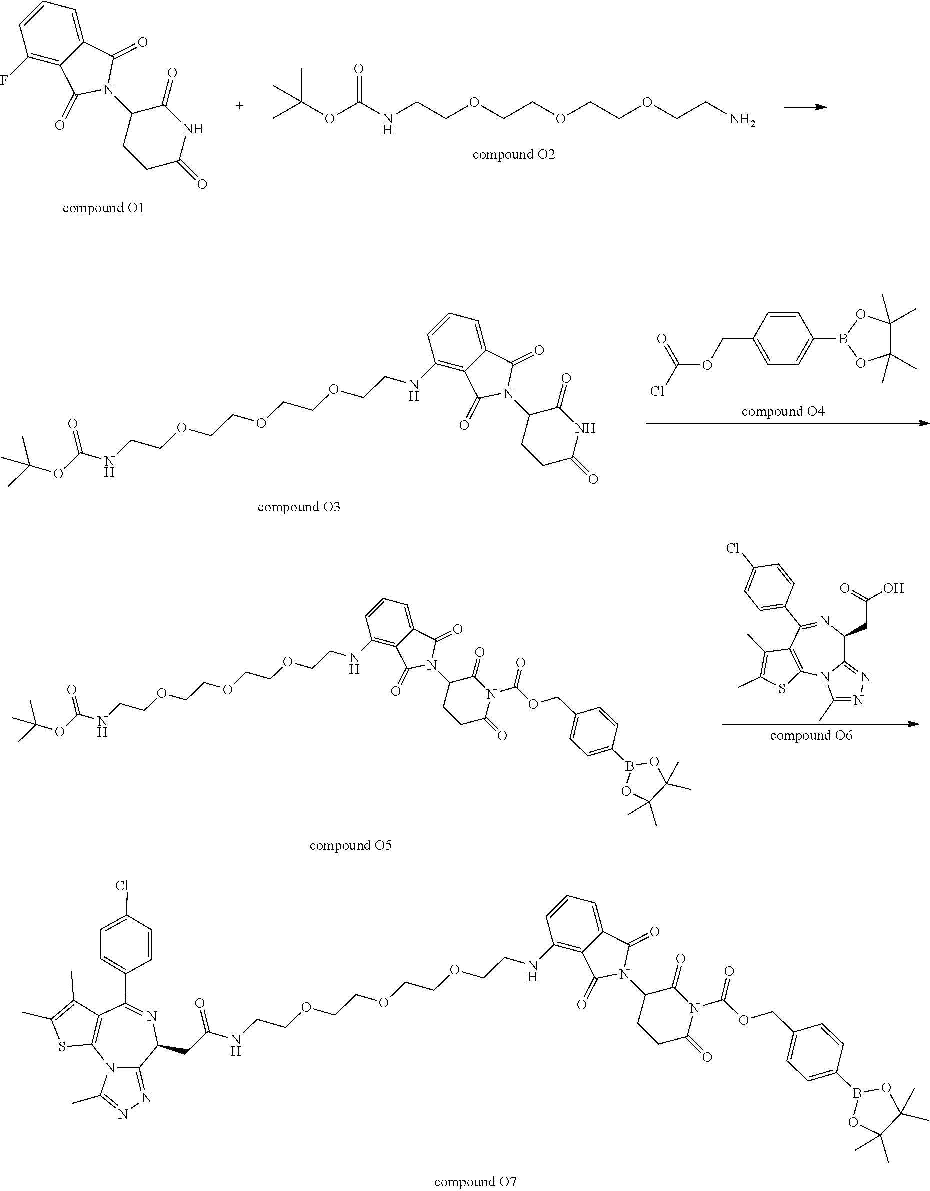

Compound O1 (1.0 g, 3.6 mmol) and compound O2 (1.6 g, 5.4 mmol) were added to a mixed solution of DMSO (15 mL) and DIPEA (5 mL). The mixture was stirred at 90° C. for 12 hours. After completion of the reaction, the mixture was diluted with EtOAc (50 mL×3) and washed with water (100 mL) followed by saturated NaCl solution (3×50 mL). The combined organic layer was dried over anhydrous Na₂SO₄, filtered, and concentrated under reduced pressure. Purification by silica gel column chromatography (CH₂Cl₂:MeOH=100:5) afforded compound O3.

¹H NMR (CDCl₃, 400 MHz) δ: 1.43 (s, 9H), 2.13-2.10 (m, 1H), 2.88-2.73 (m, 3H), 3.31 (d, J=3.88 Hz, 2H), 3.49 (q, J=5.36 Hz, 2H), 3.53 (t, J=4.64 Hz, 2H), 3.67-3.62 (m, 8H), 3.73 (t, J=5.32 Hz, 2H), 4.95-4.91 (m, 1H), 5.09 (s, 1H), 6.49 (t, J=5.28 Hz, 1H), 6.93 (d, J=8.52 Hz, 1H), 7.10 (d, J=7.04 Hz, 1H), 7.48 (t, J=7.72 Hz, 1H), 8.78 (s, 1H).

¹³C NMR (DMSO-d₆, 75 MHz) δ: 171.05, 169.28, 168.36, 167.62, 156.06, 146.86, 136.05, 132.55, 116.77, 111.66, 110.35, 79.28, 70.63, 70.20, 69.52, 48.87, 42.42, 40.35, 31.43, 28.40, 22.79.

Compound O3 (1.0 g, 1.8 mmol) was dissolved in anhydrous dichloromethane (10 mL), and the mixture was cooled to −80° C. NaHMDS (1 M in THF, 2.2 mL, 2.2 mmol) was added slowly to the mixture, and stirring was continued for 30 min. Compound O4 (0.8 g, 2.7 mmol) was dissolved in anhydrous dichloromethane (5 mL) and added slowly to the above mixture. After completion of the addition, the reaction was warmed to room temperature and stirred for 6 h. Upon reaction completion, the mixture was diluted with EtOAc (50 mL×3) and washed with water (100 mL) followed by saturated NaCl solution (50 mL×3). The combined organic layer was dried over anhydrous Na₂SO₄, filtered, and concentrated under reduced pressure. Purification by silica gel column chromatography (CH₂Cl₂:MeOH=100:5) yielded compound O5.

¹H NMR (CDCl₃, 400 MHz) δ: 1.20 (s, 12H), 1.43 (s, 9H), 2.13-2.10 (m, 1H), 2.88-2.73 (m, 3H), 3.31 (d, J=3.88 Hz, 2H), 3.49 (q, J=5.36 Hz, 2H), 3.53 (t, J=4.64 Hz, 2H), 3.67-3.62 (m, 8H), 3.73 (t, J=5.32 Hz, 2H), 4.95-4.91 (m, 1H), 5.02 (s, 2H), 5.09 (s, 1H), 6.49 (t, J=5.28 Hz, 1H), 6.93 (d, J=8.52 Hz, 1H), 7.10 (d, J=7.04 Hz, 1H), 7.28 (d, J=8.56 Hz, 2H), 7.48 (t, J=7.72 Hz, 1H), 7.78 (d, J=8.66 Hz, 2H).

¹³C NMR (DMSO-d₆, 75 MHz) δ: 171.05, 169.28, 168.36, 167.62, 156.06, 146.86, 136.12, 136.05, 133.44, 132.55, 130.45, 127.10, 116.77, 111.66, 110.35, 88.1, 79.28, 70.63, 70.20, 69.52, 66.51, 48.87, 42.42, 40.35, 31.43, 28.40, 24.72, 22.79.

Compound O5 (1.0 g, 1.2 mmol) was added slowly to a mixed solution of CH₂Cl₂ (5 mL) and CF₃COOH (1 mL). The mixture was stirred at room temperature for 6 hours. The solvent was then evaporated under reduced pressure. The resulting crude product, compound O6 (560 mg, 1.4 mmol), DIPEA (931 mg, 7.2 mmol), HATU (532 mg, 1.4 mmol), and DMAP (171 mg, 1.4 mmol) were slowly added to a solution of DMF (10 mL). The mixture was stirred at room temperature for 24 hours. After completion of the reaction, the mixture was diluted with EtOAc (50 mL×3) and washed with water (100 mL) followed by saturated NaCl solution (50 mL×3). The organic layer was dried over anhydrous Na₂SO₄, filtered, and concentrated under reduced pressure. Purification by silica gel column chromatography (CH₂Cl₂:MeOH=100:5) afforded compound O7.

¹H NMR (CDCl₃, 400 MHz) δ: 1.20 (s, 12H), 2.13-2.10 (m, 1H), 2.14 (s, 3H), 2.35 (s, 3H), 2.37 (s, 3H), 2.92 (m, 2H), 2.88-2.73 (m, 3H), 3.31 (d, J=3.88 Hz, 2H), 3.49 (q, J=5.36 Hz, 2H), 3.53 (t, J=4.64 Hz, 2H), 3.67-3.62 (m, 8H), 3.73 (t, J=5.32 Hz, 2H), 4.72 (m, 1H), 4.95-4.91 (m, 1H), 5.02 (s, 2H), 5.09 (s, 1H), 6.49 (t, J=5.28 Hz, 1H), 6.93 (d, J=8.52 Hz, 1H), 7.10 (d, J=7.04 Hz, 1H), 7.28 (d, J=8.56 Hz, 2H), 7.48 (t, J=7.72 Hz, 1H), 7.51 (d, J=8.04 Hz, 2H), 7.78 (d, J=8.66 Hz, 2H), 7.89 (d, J=8.12 Hz, 2H).

¹³C NMR (DMSO-d₆, 75 MHz) δ: 171.05, 169.28, 168.66, 168.36, 167.62, 156.06, 155.62, 149.36, 146.86, 137.12, 136.67, 136.12, 136.05, 136.03, 133.95, 133.44, 132.55, 131.42, 130.66, 130.45, 128.95, 127.10, 120.03, 116.77, 111.66, 110.35, 88.1, 70.63, 70.20, 69.52, 66.51, 55.21, 48.87, 42.13, 42.42, 40.35, 31.43, 24.72, 22.79, 12.25, 10.53, 10.45.

### Synthesis of Compound P2

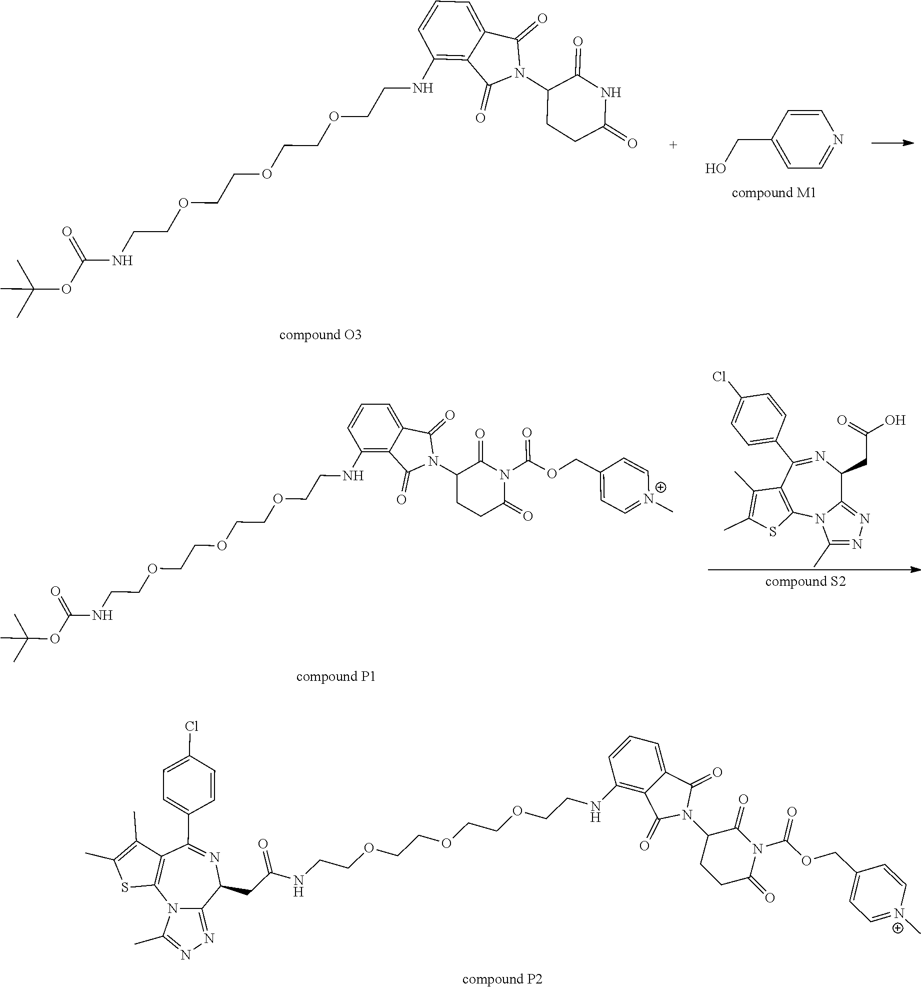

Compound O3 (1.0 g, 1.8 mmol) was dissolved in anhydrous dichloromethane (10 mL), and the mixture was cooled to −80° C. NaHMDS (1 M in THF, 2.2 mL, 2.2 mmol) was added slowly to the mixture, and stirring was continued for 30 min. Compound M1 (294 mg. 2.7 mmol) and triphosgene (267 mg, 0.9 mmol) were dissolved in anhydrous dichloromethane (2 mL), and the resulting solution was added slowly to the above mixture after 30 min. Upon completion of the addition, the reaction was warmed to room temperature and stirred for 6 h. After the reaction was complete, the mixture was diluted with EtOAc (50 mL×3) and washed with water (100 mL) followed by saturated NaCl solution (50 mL×3). The organic layer was dried over anhydrous Na₂SO₄, filtered, and concentrated under reduced pressure. Purification by silica gel column chromatography (CH₂Cl₂:MeOH=100:5) afforded compound P1.

¹H NMR (CDCl₃, 400 MHz) δ: 1.43 (s, 9H), 2.13-2.10 (m, 1H), 2.88-2.73 (m, 3H), 3.31 (d, J=3.88 Hz, 2H), 3.49 (q, J=5.36 Hz, 2H), 3.53 (t, J=4.64 Hz, 2H), 3.67-3.62 (m, 8H), 3.73 (t, J=5.32 Hz, 2H), 4.38 (s, 3H), 4.95-4.91 (m, 1H), 5.02 (s, 2H), 5.09 (s, 1H), 6.49 (t, J=5.28 Hz, 1H), 6.93 (d, J=8.52 Hz, 1H), 7.10 (d, J=7.04 Hz, 1H), 7.48 (t, J=7.72 Hz, 1H), 8.22 (d, J=6.36 Hz, 2H), 9.01 (d, J=6.56 Hz, 2H).

¹³C NMR (DMSO-d₆, 75 MHz) δ: 171.05, 169.28, 168.36, 167.62, 159.23, 156.06, 154.23, 146.86, 146.53, 136.05, 132.55, 128.91, 116.77, 111.66, 110.35, 79.28, 70.63, 70.20, 69.52, 66.51, 49.03, 48.87, 42.42, 40.35, 31.43, 28.40, 22.79.

Compound P1 (1.0 g, 1.4 mmol) was added slowly to a mixed solution of CH₂Cl₂ (5 mL) and CF₃COOH (1 mL). The mixture was stirred at room temperature for 6 hours. The solvent was then evaporated under reduced pressure. The resulting product, compound S2 (680 mg, 1.7 mmol), DIPEA (931 mg, 7.2 mmol), HATU (646 mg, 1.7 mmol), and DMAP (208 mg, 1.7 mmol) were slowly added to a solution of DMF (10 mL). The mixture was stirred at room temperature for 24 hours. After completion of the reaction, the mixture was diluted with EtOAc (50 mL×3) and washed with water (100 mL) followed by saturated NaCl solution (50 mL×3). The organic layer was dried over anhydrous Na₂SO₄, filtered, and concentrated under reduced pressure. Purification by silica gel column chromatography (CH₂Cl₂:MeOH=100:5) afforded compound P2.

¹H NMR (CDCl₃, 400 MHz) δ: 2.13-2.10 (m, 1H), 2.14 (s, 3H), 2.35 (s, 3H), 2.37 (s, 3H), 2.92 (m, 2H), 2.88-2.73 (m, 3H), 3.31 (d, J=3.88 Hz, 2H), 3.49 (q, J=5.36 Hz, 2H), 3.53 (t, J=4.64 Hz, 2H), 3.67-3.62 (m, 8H), 3.73 (t, J=5.32 Hz, 2H), 4.38 (s, 3H), 4.72 (m, 1H), 4.95-4.91 (m, 1H), 5.02 (s, 2H), 5.09 (s, 1H), 6.49 (t, J=5.28 Hz, 1H), 6.93 (d, J=8.52 Hz, 1H), 7.10 (d, J=7.04 Hz, 1H), 7.28 (d, J=8.56 Hz, 2H), 7.48 (t, J=7.72 Hz, 1H), 7.51 (d, J=8.04 Hz, 2H), 7.78 (d, J=8.66 Hz, 2H), 7.89 (d, J=8.12 Hz, 2H).

¹³C NMR (DMSO-d₆, 75 MHz) δ: 171.05, 169.28, 168.66, 168.36, 167.62, 156.06, 155.62, 149.36, 146.86, 137.12, 136.67, 136.12, 136.05, 136.03, 133.95, 133.44, 132.55, 131.42, 130.66, 130.45, 128.95, 127.10, 120.03, 116.77, 111.66, 110.35, 70.63, 70.20, 69.52, 66.51, 55.21, 49.03, 48.87, 42.13, 42.42, 40.35, 31.43, 22.79, 12.25, 10.53, 10.45.

### TEST EXAMPLE

### Test Example 1: In Vitro Anti-Tumor Effects of Protein Degradation Chimeras of all Embodiments of the Present Disclosure

Experimental Procedure: MCF-7 cells in the logarithmic growth phase were used for the experiment. The cells were seeded at 5000 cells per well in 96-well plates and incubated for 24 hours at 37° C. The cells were treated with Example compounds A1-D3 for 3 h. Subsequently, the irradiated group was irradiated with 10-30 Gy of X-rays, and the cells were continued to be incubated until 72 h after completion of irradiation. At the end of drug treatment, 20 μL of CellTiter 96° solution was added to each well. The well plates were incubated in an incubator for 2-4 hours, and then the absorbance was measured using a plate reader. Data were analyzed with GraphPad Prism software to obtain the IC₅₀ values of each test compound.

The experimental results are shown in Table 11.

TABLE 11

MCF-7

| | | Irradiation with |
|---|---|---|
| | Radiation-free | 10 Gy or 30 Gy |
| Compound No. | IC₅₀ (μM) | IC₅₀ (μM) |
| A1 | 1.019 | 0.122 |
| B1 | 1.078 | 0.119 |
| C1 | 0.964 | 0.146 |
| D1 | 1.219 | 0.152 |
| A2 | 1.106 | 0.103 |
| B2 | 1.056 | 0.119 |
| C2 | 0.984 | 0.107 |
| D2 | 1.006 | 0.127 |
| A3 | 2.539 | 0.273 |
| B3 | 2.764 | 0.338 |
| C3 | 1.861 | 0.97 |
| D3 | 1.963 | 0.104 |

From the above table, none of the compounds A1-D3 had significant inhibitory activity on MCF-7 cells when they were not irradiated. However, when the cells were co-treated with X-ray irradiation and the above drugs, the irradiation induced the breakage of the X-ray responsive group, releasing PROTAC molecules, which resulted in significant cytotoxicity.

### Test Example 2: Verification of Radiation Response of Compound N2

An appropriate amount of N2 was weighed and dissolved in DMSO to prepare a 10 mM stock solution. Prior to irradiation, the stock solution was diluted to 10 M using 0.02 M PB buffer. Argon gas was slowly purged into the solution to ensure complete oxygen removal. The sealed samples were then irradiated in an irradiator, and the fluorescence intensity of the compound was measured post-irradiation.

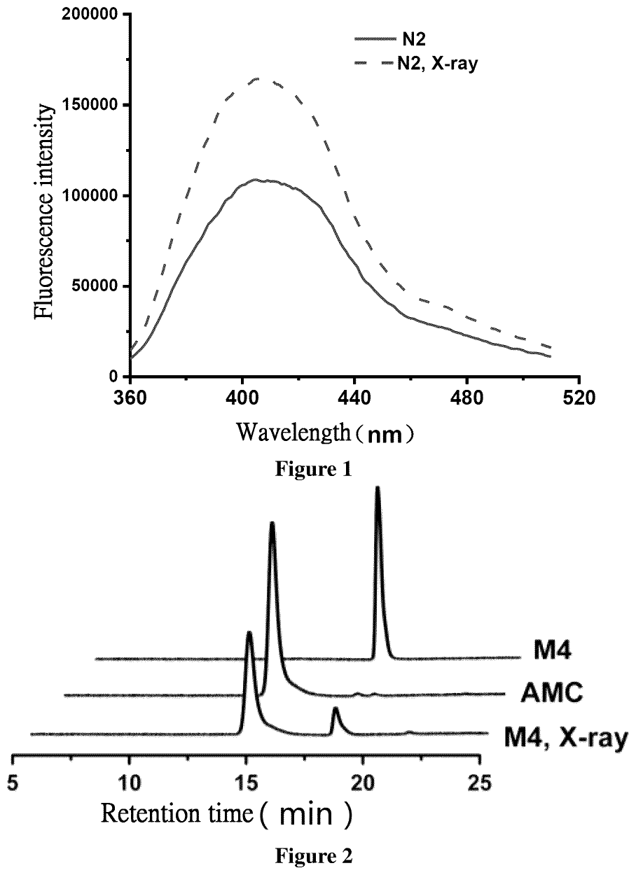

As shown in FIG. 1, X-ray irradiation induced a chemical cleavage reaction in N2, releasing a fluorophore and consequently causing a significant enhancement in fluorescence signal. The observed fluorescence intensity changes demonstrate that N2 possesses excellent radiation-responsive capability.

### Test Example 3: Verification of Radiation Response of Compound M4

An appropriate amount of M4 was weighed and dissolved in DMSO to prepare a 10 mM stock solution. Prior to irradiation, the stock solution was diluted to 10 M using 0.02 M PB buffer. Argon gas was slowly purged into the solution to ensure complete oxygen removal. The sealed samples were then irradiated in an irradiator, and the radiation-responsive capability of M4 was analyzed by high-performance liquid chromatography (HPLC) post-irradiation.

As shown in FIG. 2, X-ray irradiation induced a chemical reaction in M4, releasing a fluorophore. These results demonstrate that M4 exhibits excellent radiation-responsive properties.

### Test Example 4: Protein Degradation Activity Testing of PROTAC Drug AS2 and Radiation-Responsive PROTAC Prodrug A1

Cell treatment: MCF-7 cells in the logarithmic growth phase were taken for the experiment. Cells were inoculated in six-well plates at a density of 5.0×10⁵ cells/per well and incubated at 37° C. for 24 hours. Cells were treated with Example compounds A1-D3 for 3 h, followed by irradiation using 10 Gy or 30 Gy X-rays, and the cells were continued to be incubated up to 24 h after completion of irradiation.

Whole cell protein extraction: After completion of drug treatment, the culture medium was discarded, and the cells were washed 3 times with pre-cooled PBS. Cells were scraped off using a cell scraper, and the resulting solution was carefully aspirated and centrifuged at 300 g for 5 min at 4° C. to obtain cells, which were washed with PBS, resuspended, and centrifuged under the same conditions.

Cell lysis: Cells were washed with pre-cooled PBS and lysed in pre-cooled lysis buffer (containing 1% protease and phosphatase inhibitor) for 30 min, and cells were vigorously vortexed several times during lysis. After lysis was completed, the cells were centrifuged at 11,000 g for 15 min at 4° C., and the supernatant was collected as whole cell protein.

Protein denaturation: protein concentration was measured by Bradford assay. Proteins were diluted using 4× Protein Sampling Buffer. Subsequently, the proteins were denatured by heating in a metal bath at 100° C. for 15 min.

### Protein Immunoblotting Assay (Western Blotting, WB) was Performed to Quantify BRD4 Protein:

1) Protein electrophoresis: proteins were separated in 4-12% or 4-20% Bis-Tris SDS-PAGE gels. The appropriate volume of the above cell whole protein extract was added to the lanes of the above SDS-PAGE gel. Turn on the power and electrophoresis at 120 V for 15 minutes, then adjust the voltage to 180 V and continue electrophoresis until the loading buffer reaches the lowest end of the gel.

(2) Membrane transfer: After electrophoresis, remove the gel, cut a PVDF membrane (0.45 m) of the appropriate size and activate the membrane with anhydrous methanol, and install the membrane transfer device in the following order from the negative pole to the positive pole: sponge cushion, gel, PVDF membrane, sponge cushion. After installing the membrane transfer device, place it in the corresponding membrane transfer tank, check the liquid level of the solution required for membrane transfer, turn on the power, and complete the membrane transfer after 3 transfer cycles.

3) Blocking: Place the membrane in QuickBlock™ Western Blocking Buffer and shake it for 20 minutes at room temperature.

4) Primary antibody incubation: After the blocking, discard the blocking solution, add the primary antibody diluted to the appropriate concentration, and gently shake the membrane overnight at 4° C. After completing the incubation, wash the membrane 4 times with 1×TBST for 5 min/time.

(5) Secondary antibody incubation: Add secondary antibody diluted to the appropriate concentration and gently shake the PVDF membrane for 1 hour at room temperature. After incubation, the membrane was washed 4 times with 1×TBST for 5 min/time.

6) Developing: Prepare the ECL developing solution before developing. Lay the PVDF membrane flat on a clean plastic wrap, add the developer solution to the membrane, and let it stand for 1 minute away from light. Subsequently, the PVDF membrane was imaged on a gel system.

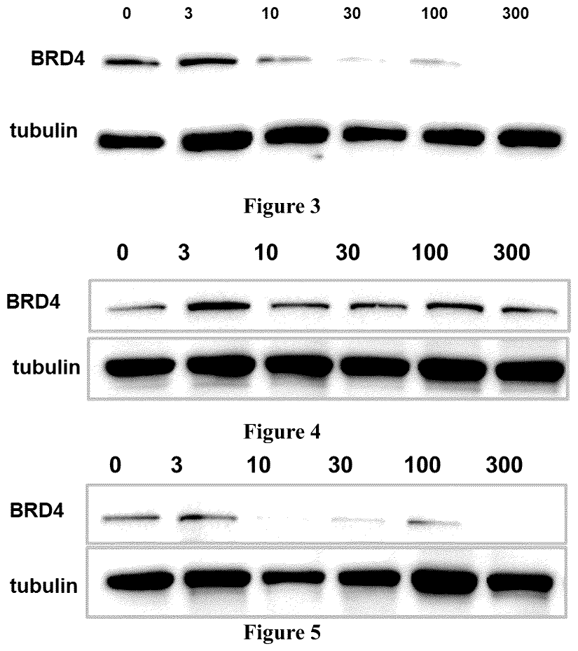

The degradation activity of the PROTAC drug AS2 on BRD4 in this example is as follows: In the breast cancer cell line MCF-7, WB results demonstrated that the PROTAC drug AS2 effectively degraded BRD4 at tested concentrations (0-300 nM), with a DC50 of 31.23 nM, as shown in FIG. 3.

Similarly, the degradation activity of the X-ray-responsive protein degradation chimeric prodrug A1 on BRD4 in this example is as follows: In the MCF-7 breast cancer cell line, WB results demonstrated that the PROTAC prodrug, prior to activation, failed to degrade BRD4 at all tested concentrations (0-300 nM), as shown in FIG. 4. In contrast, when MCF-7 cells were co-treated with X-ray irradiation and the prodrug, BRD4 protein was significantly degraded, with as low as 10 nM prodrug achieving >50% degradation, as shown in FIG. 5.

### Test Example 5: Annexin V/PI Double Staining for Apoptosis Detection

The surface of normal cells consists of lipids distributed asymmetrically on the inner and outer leaflets of the plasma membrane. One of them, phosphatidylserine (PS), is normally distributed only on the inner leaflet of the plasma membrane, and is therefore exposed only to the cytoplasm. However, when cells undergo apoptosis, PS is exposed on the outer leaflets of the plasma membrane. Annexin V is a phospholipid-binding protein capable of binding to PS. Therefore, fluorescently labeled Annexin V can be used to detect PS exposed to the outer leaflets of early apoptotic cells. By co-staining with propidium iodide (PI), Annexin V-PI can distinguish normal living cells, early apoptotic cells, and late apoptotic cells.

Cell treatment: MCF-7 cells in the logarithmic growth phase were taken for the experiment. Cells were inoculated in six-well plates at a density of 5.0×10⁵ cells/per well and incubated at 37° C. for 24 hours. Cells were treated with Example PROTAC drug AS2 and PROTAC prodrug A1 for 3 h, followed by X-ray irradiation, and cells continued to be cultured until 48 h after completion of irradiation.

Cell collection: Collect the culture medium in a 1.5 mL centrifuge tube for spare. The cells were washed 3 times with pre-cooled PBS, followed by digestion of the cells with 0.25% EDTA-free trypsin at 37° C. and making a suspension of individual cells. The supernatant was discarded after centrifugation at 300 g for 5 min at 4° C. The cells were resuspended using the collected medium, and the supernatant was discarded by centrifugation again under the same conditions.

Cell staining: Staining was performed according to the Annexin V/PI Double Staining Kit instructions. 1×10⁵ cells were resuspended in 200 μL Binding Buffer, and 4 μL of 0.5 mg/mL PI and 2 μL of Annexin V-FITC solution were added. After incubation for 15 min at room temperature away from light, fluorescence detection was carried out by flow cytometry.

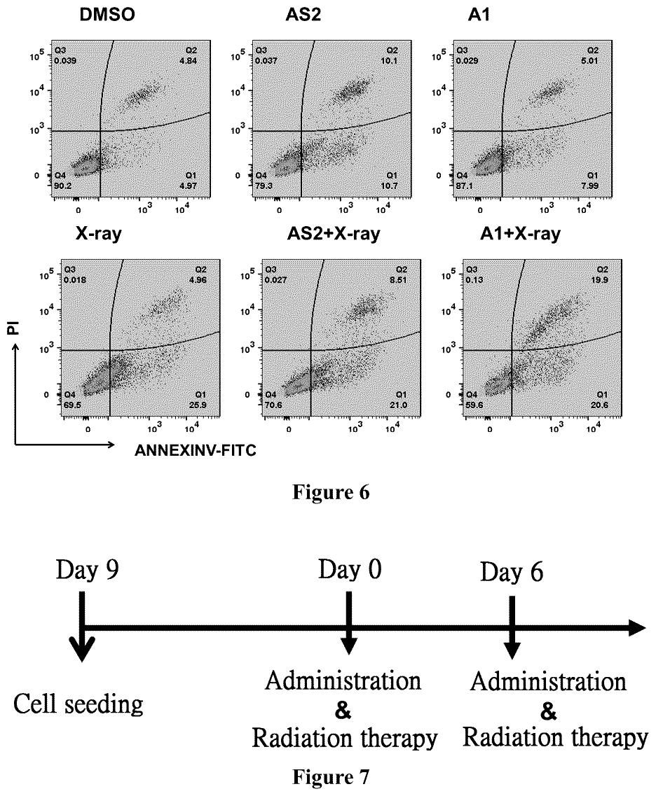

The test results are shown in FIG. 6.

As shown in FIG. 6, according to the principle of Annexin V/PI double staining reagent action, the scatter plot was divided into four quadrants, with the lower left quadrant being negative for both FITC/PI, representing living cells; the lower right quadrant being positive for FITC and negative for PI, representing early apoptotic cells; and the upper right quadrant being positive for both FITC/PI, representing necrotic cells or late apoptotic cells. It is observed that the ability of compound A1 to induce MCF-7 apoptosis changed significantly before and after irradiation. The ability of A1 without X-ray irradiation to induce apoptosis was comparable to that of control DMSO, with no significant cell killing ability. However, when X-rays and A1 were synergistically treated with MCF-7 cells, significantly elevated early apoptotic and late apoptotic cells were detected. The results suggest that compound A1 can be activated intracellularly by X-rays, which in turn induces apoptosis.

### Test Example 6: In Vivo Anti-Tumor Activity Studies of AS2 and A1 Molecules

To investigate the in vivo activation and antitumor activity of the PROTAC compound AS2 and X-ray-responsive PROTAC compound A1, we performed in vivo antitumor activity studies. The MCF-7 cell line was selected, and experiments were performed in BALB/c nude mice.

Experimental animals: BALB/c nude mice (female; 6-8 weeks old) were all purchased from Beijing Vital River Laboratory Animal Technology Co., Ltd and housed in pathogen-free conditions with standard temperature and humidity at the Laboratory Animal Resources Center of Tsinghua University. All animal experiments were approved by the Animal Ethics Committee of Tsinghua University.

Tumor model establishment: MCF-7 cells in logarithmic growth phase were taken, and 0.25% trypsin was added to digest the cells after washing the cells for 3 times with pre-cooled PBS buffer. The digested cells were centrifuged at 1000 rpm/min for 5 min, the precipitate was discarded, and the cells were resuspended with pre-cooled PBS and carefully blown to make a single-cell solution. After the cells were counted by a counter, the cell density was adjusted to 1×10⁷ cells/mL, and 10 mL was taken into a centrifuge tube and stored on ice. Before inoculation of cancer cells, nude mice were gently wiped under the skin of the right axilla with a paper towel sprayed with 75% alcohol. A 1 mL disposable syringe was used to aspirate 200 μL of cell suspension, and the air in the needle was removed, and a single subcutaneous injection was carried out by puncturing about 1 cm forward from the needle inlet site. The needle was withdrawn slowly to avoid fluid leakage. Observe the tumor growth status daily.

Drug administration: When the tumor volume reached 120 mm³, the tumor-bearing mice were randomly grouped. Six groups were set up, namely PBS, PROTAC drug AS2, PROTAC prodrug A1, PBS+X-ray irradiation, PROTAC drug AS2+X-ray irradiation, and PROTAC prodrug A1+X-ray irradiation. Each drug was dispersed in the following solution: 1.25% DMSO+20% PEG+5% Tween 80+73.75% dd H₂O; administered dose: 10 mg/kg; subcutaneous injection. X-ray irradiation was performed 3 hours after drug injection. The irradiation dose was 5 Gy. The timeline of drug administration is shown in FIG. 7.

### Experimental Recording:

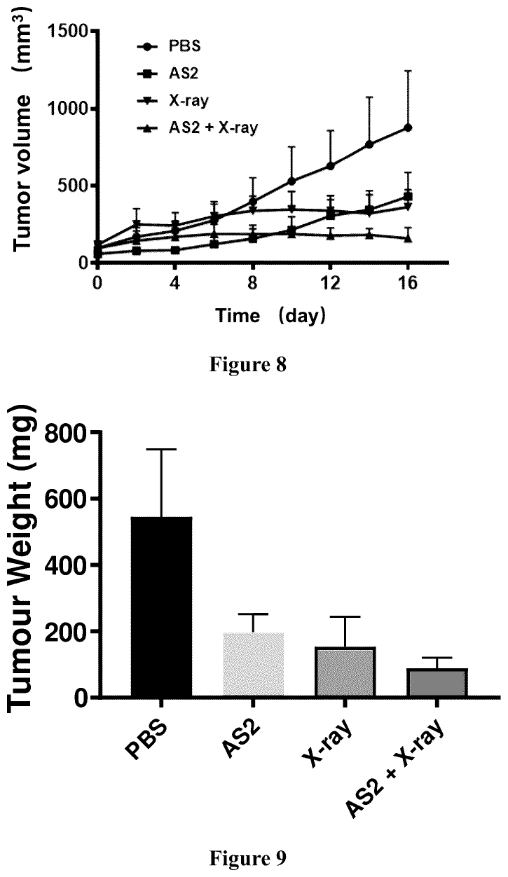

Tumor size and body weight were monitored every other day. Tumor volume was calculated using the formula: tumor volume=length×width²/2. After 16 days of monitoring, mice in each group were euthanized, and tumors and major organs were collected. The changes in tumor volume of mice in the compound AS2 group over time of administration are shown in FIG. 8.

The test results showed that the mice were in good health during the experimental period, and the mice moved, ate, and excreted normally. From the results of drug administration, compared with the PBS group, compound AS2 had a poor antitumor effect although it could inhibit tumor growth, which was mainly caused by the lower administration dose and administration frequency. On the contrary, when AS2 was combined with X-ray irradiation, tumor growth was significantly inhibited, a result suggesting that AS2 could enhance the effect of radiation therapy.

The results of tumor weight in the compound AS2 group at day 16 are shown in FIG. 9. This result also suggests that AS2 can significantly enhance the effect of radiation therapy. After two synergistic administrations of AS2 and radiation therapy, there was essentially no tumor growth.

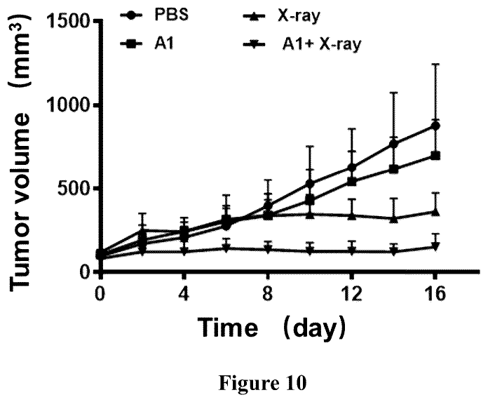

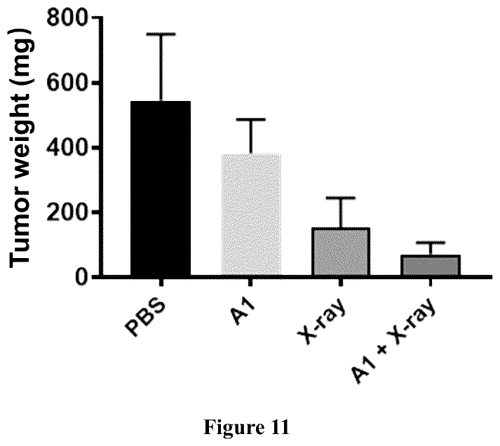

Tumor volume changes over treatment time in the compound A1 group was shown in FIG. 10, and tumor weight in the compound A1 group on day 16 was shown in FIG. 11. The experimental results showed that the PROTAC-responsive compound A1 failed to inhibit the tumor growth, which was mainly due to the loss of protein-targeting degradation ability of the prodrug after chemical modification of the “cage”. However, after X-ray irradiation to the tumor site in mice, the prodrug in the tumor site responded to the X-rays and released the irradiation-responsive “chemical cage” and PROTAC molecules efficiently. The released PROTAC molecules act as catalysts to degrade target proteins in the tumor microenvironment and enhance the efficacy of radiation therapy.

1. A compound shown in formula (I) or an isomer or a pharmaceutically acceptable salt or deuteride thereof: 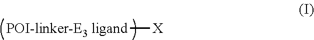
wherein POI is selected from any target protein ligand of interest; E3 ligand is an E3 ubiquitin ligase ligand; linker is a functional group linking the target protein ligand and the E3 ubiquitin ligase ligand; and X is any functional molecule responsive to irradiation.

2. The compound according to claim 1 or an isomer or a pharmaceutically acceptable salt or deuterium substituent thereof, wherein X is structured as shown in formula (I-1) below: 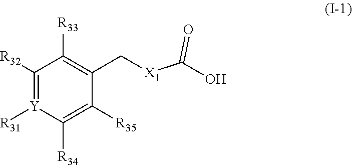
wherein,
R₃₁ is selected from —N₃, H, C1-10 alkyl, or 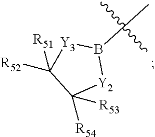
R₃₂ and R₃₄ are each independently selected from H, halogen, or —R₄₁—Y₁—R₄₂;
R₃₃ and R₃₅ are each independently selected from F, Cl, Br, I, or H;
Y₁ is selected from O or S;
R₄₁ is selected from C1-10 alkylene; R₄₂ is selected from C1-10 alkyl;
R₅₁, R₅₂, R₅₃, and R₅₄ are each independently selected from H or C1-10 alkyl;
Y₂ and Y₃ are each independently selected from O or S;
X₁ is selected from O, S, or NH;
Y is selected from C or N⁺.

3. The compound according to claim 2 or an isomer or a pharmaceutically acceptable salt or deuterium substituent thereof, wherein,
R₃₁ is selected from 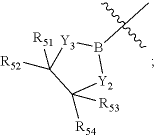
R₃₂, R₃₃, R₃₄ and R₃₅ is selected from H or F, Cl, Br, I;
X₁ is selected from O or S;
Y is C.

4. The compound according to claim 2 or an isomer or a pharmaceutically acceptable salt or deuterium substituent thereof, wherein,
R₃₁ is selected from C1-10 alkyl;
R₃₂, R₃₃, R₃₄ and R₃₅ is selected from H or F, Cl, Br, I;
X₁ is selected from O or S;
Y is selected from N⁺.

5. The compound according to claim 1 or an isomer or a pharmaceutically acceptable salt or deuterium substituent thereof, wherein X is structured as shown in formula (II) below: 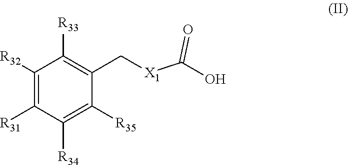
wherein R₃₁ is selected from —N₃ or H;
R₃₂ and R₃₄ are selected from halogen or —R₄₁—Y₁—R₄₂;
R₃₃ and R₃₅ are selected from F, Cl, Br, I or H;
Y₁ is selected from O or S;
R₄₁ is selected from C1-10 alkylidene; R₄₂ is selected from C1-10 alkyl;
X₁ is selected from O, S or NH.

6. The compound or isomer or pharmaceutically acceptable salt or deuteride thereof according to claim 2, wherein X is structured as shown in formula (II-1), formula (II-2), formula (II-3) or formula (II-4) below:

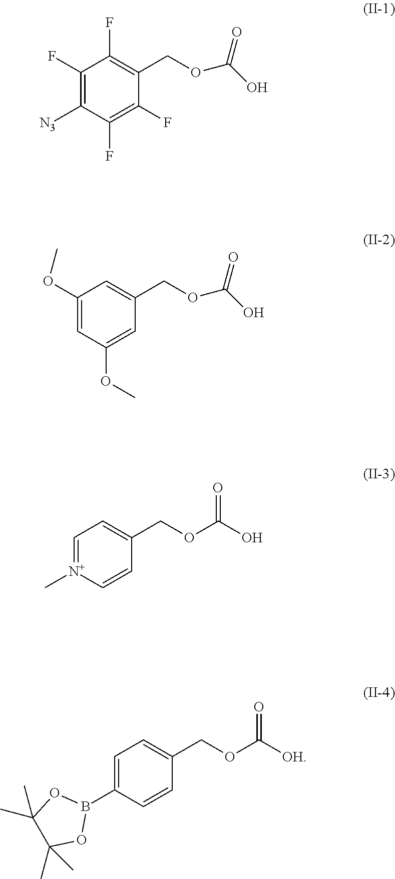 [SMILES: [N-]=[N+]=Nc1c(F)c(F)c(COC(=O)O)c(F)c1F ; COc1cc(COC(=O)O)cc(OC)c1 ; C[n+]1ccc(COC(=O)O)cc1 ; CC1(C)OB(c2ccc(COC(=O)O)cc2)OC1(C)C]

7. The compound according to claim 1 or an isomer or a pharmaceutically acceptable salt or deuterium substitute, wherein the POI ligand is a BET family protein ligand JQ1 and its derivative.

8. The compound according to claim 7 or an isomer or a pharmaceutically acceptable salt or deuterium substitute thereof, wherein the POI ligand has the structure shown in formula (III) below:

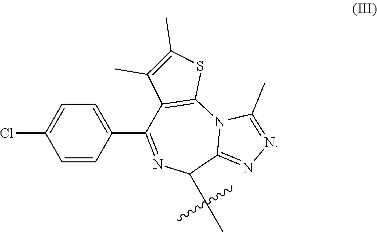 [SMILES: *C1N=C(c2ccc(Cl)cc2)c2c(sc(C)c2C)-n2c(C)nnc21]

9. The compound according to claim 1 or an isomer or pharmaceutically acceptable salt or deuteride thereof, wherein the linker structure is:
—R₁₇—(C═O)ₘ—NH—R₁₁—(R₁₅)ₐ—(NR₂₁)_{b}—X₂—R₁₂-(A)_{c}-R₁₃—(CH₂—O—X₃)_{d}—Y—R₁₄—(R₁₆)ₑ—
R₁₁ and R₁₄ are each independently selected from C1-10 alkylidene; R₁₂, R₁₃ and R₁₇ are C1-10 alkylidene or a bond;
R₁₅ and R₁₆ are each independently —(C═O)—;
R₂₁ is selected from H or an alkyl group of C1-10;
A is selected from a C4-12 heterocyclylene group or a C5-10 heteroarylene group, said heterocyclylene group or heteroarylene group containing 1-4 heteroatoms selected from N, O or S;
X₂, Y are each independently —O—, —S— or a bond;
X₃ is a bond or —CH₂—;
a, b, c, e is 0 or 1; d is 0, 1, 2, 3, 4, 5, 6 or 7; m and n are each independently 0 or 1; preferably, said heteroarylene group is selected from one of the following structures:
thienylidene, furylidene, pyrrolidene, isothiazolylidene, isoxazolylidene, pyrazolylidene, thiadiazolylidene, oxadiazolylidene, thiadiazolylidene, oxadiazolylidene, tetrazolylidene, triazolylidene, pyridylidene, pyrimidylidene, pyridazinylidene, pyrazinylidene, triazinylidene;
preferably, the heterocyclylene group is selected from one of the following structures:
azetidine, azolidine, azepane, azocane, azonane, oxetane, oxolane, oxane, oxepane, oxocane, thietane, thiolane, thiane, thiepane, thiocane, diazetidine, diazolidine, diazepane, diazocane, diazonane, dioxetane, dioxolane, dioxane, dioxepane, dioxocane, dithietane, dithiolane, dithiane, dithiepane, dithiocane, and

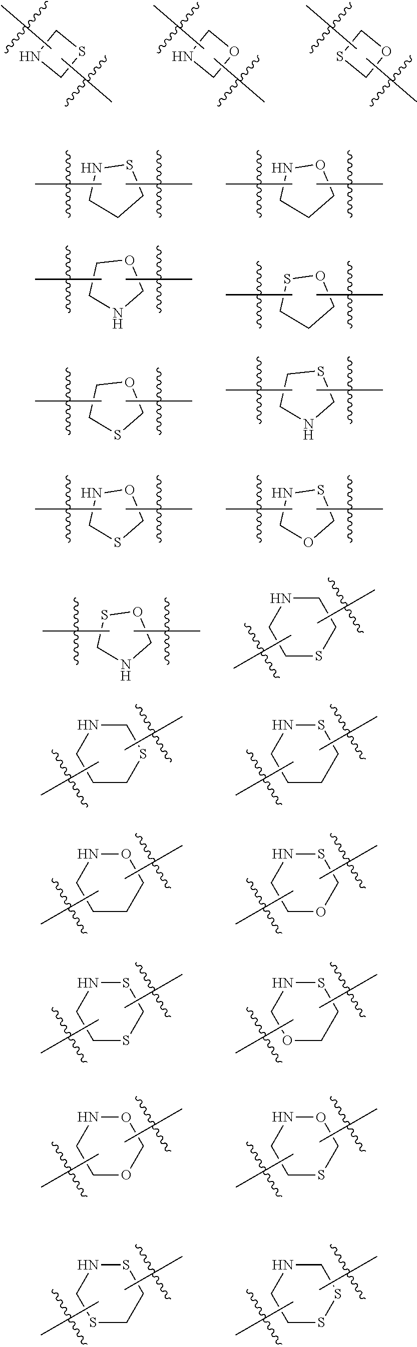

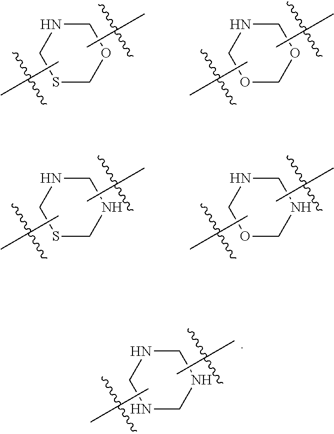

10. The compound according to claim 9 or an isomer or pharmaceutically acceptable salt or deuteride thereof, wherein the linker structure is selected from one of the following structures:
—(C═O)—NH—R₁₁—(CH₂—O—CH₂)_{d1}—R₁₄—,
—(C═O)—NH—R₁₁—(R₁₅)ₐ—(NR₂₁)—R₁₂—(CH₂—O—CH₂)_{d2}—R₁₄—,
—(C═O)—NH—R₁₁—X—R₁₂-A-R₁₃—(CH₂—O—CH₂)_{d2}—Y—R₁₄—(R₁₆)ₑ—,
—(C═O)—NH—R₁₁—R₁₂—R₁₃—R₁₄—; or
—R₁₇—(C═O)—NH—R₁₁—R₁₄—NH—(C═O)—
wherein R₁₁, R₁₂, R₁₃ and R₁₄ are each independently selected from alkylidene of C1-4; R₂₁ is H or alkyl of C1-4; d1 is 1, 2, 3, 4 or 5; d2 is 0, 1, 2, 3 or 4.

11. The compound according to claim 9 or an isomer or pharmaceutically acceptable salt or deuteride thereof, wherein the linker structure is selected from one of the following structures: 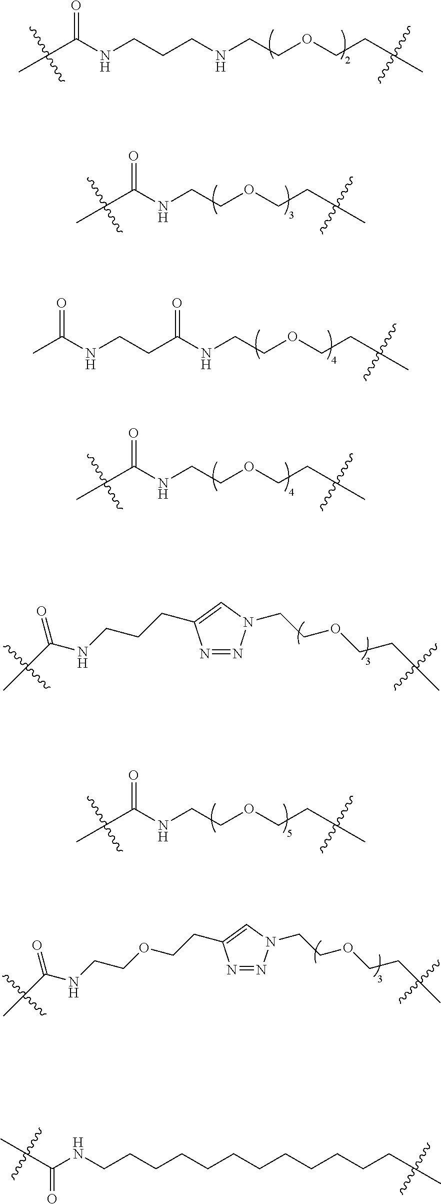 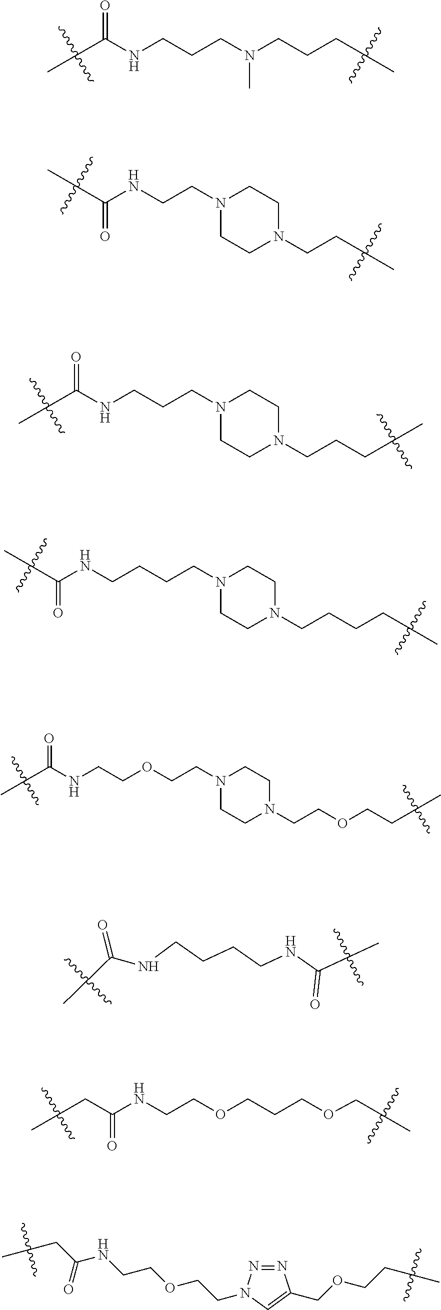 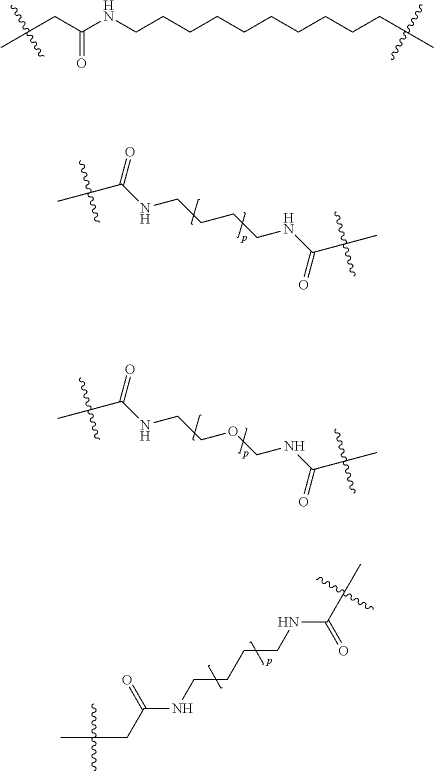
where p is 1, 2, 3, 4 or 5.

12. The compound according to claim 1, or an isomer or a pharmaceutically acceptable salt or deuterated substitute thereof, wherein the E3 ligand is selected from the ligands of the E3 ligases of VHL, CRBN, GID4, DCAF2, KLHL41, SPSB4, TRIM7, TRIM9, RNF43 and RNF182.

13. The compound according to claim 1 or an isomer or pharmaceutically acceptable salt or deuteride thereof, wherein said E3 ligand has the structure shown in the following formula (IV-1) or formula (IV-2): 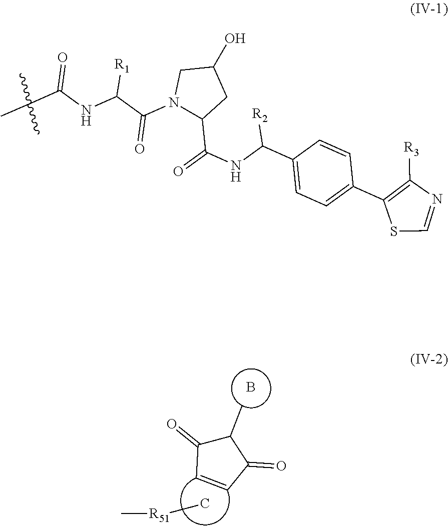
wherein R1, R2 and R3 are each independently selected from an alkyl group of C1-10; optionally, said alkyl group is further substituted with H, halogen, hydroxyl, carboxyl, amino, cyano, carbonyl or alkyl group of C1-5;
ring B is a heterocycloalkyl group of C4-10; said heterocycloalkyl group is optionally substituted with 0, 1, 2, 3 or 4=0; said heterocycloalkyl group contains 1, 2, 3 or 4 heteroatoms selected from N, O, and S and contains at least one N atom;
ring C is an aromatic ring of C6-12; said aromatic ring is optionally substituted with 0, 1, 2, 3, 4, 5 or 6 substituents selected from F, Cl, Br, I, C1-6 alkyl, hydroxy, amino, cyano and nitro;
R51 is selected from —O— or —NH—.

14. The compound according to claim 1, or an isomer or a pharmaceutically acceptable salt or deuterium substituent thereof, wherein X is attached to the E3 ligand structure or to the linker.

15. The compound or isomer or pharmaceutically acceptable salt or deuterium substitute thereof according to claim 1, wherein X is attached to the hydroxyl or amino group of the E3 ligand or linker structure.

16. The compound or isomer or pharmaceutically acceptable salt or deuterium substitute thereof according to claim 1, wherein said compound is structured as shown in the following formulae (V-1), (V-2), (V-3) and (V-4):

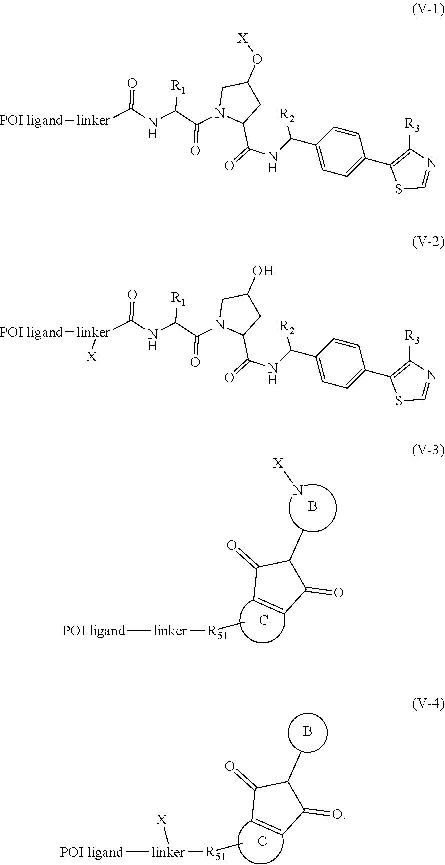

17. The compound according to claim 13 or an isomer or pharmaceutically acceptable salt or deuterated substitute thereof, wherein R₁, R₂, and R₃ are each independently selected from methyl, ethyl, n-propyl, isopropyl, n-butyl, iso-butyl, tert-butyl, n-amyl, iso-pentyl, neo-pentyl, 2-methylbutyl, 3-methylbutyl, n-hexyl, 2-methylpentyl, 3-methylpentyl 4-methylpentyl, 2-ethylbutyl or 3-ethylbutyl;
Ring B is a heterocycloalkyl group selected from the following structures: 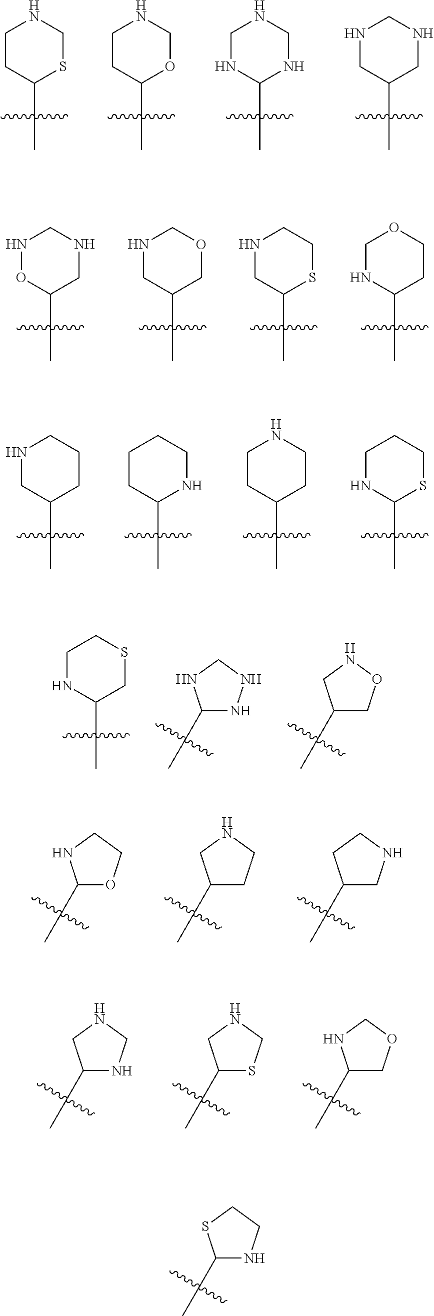
said heterocycloalkyl is optionally substituted with 0, 1, 2, 3 or 4 ═O;
ring C is an aromatic ring selected from the following structures: 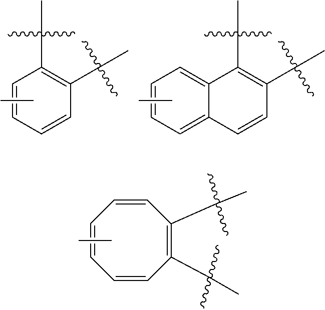
said aromatic ring is optionally substituted with 0, 1, 2, 3, 4, 5 or 6 substituents selected from F, Cl, Br, I, C1-6 alkyl, hydroxy, amino, cyano and nitro;
preferably,
ring B is selected from heterocycloalkyl as follows: 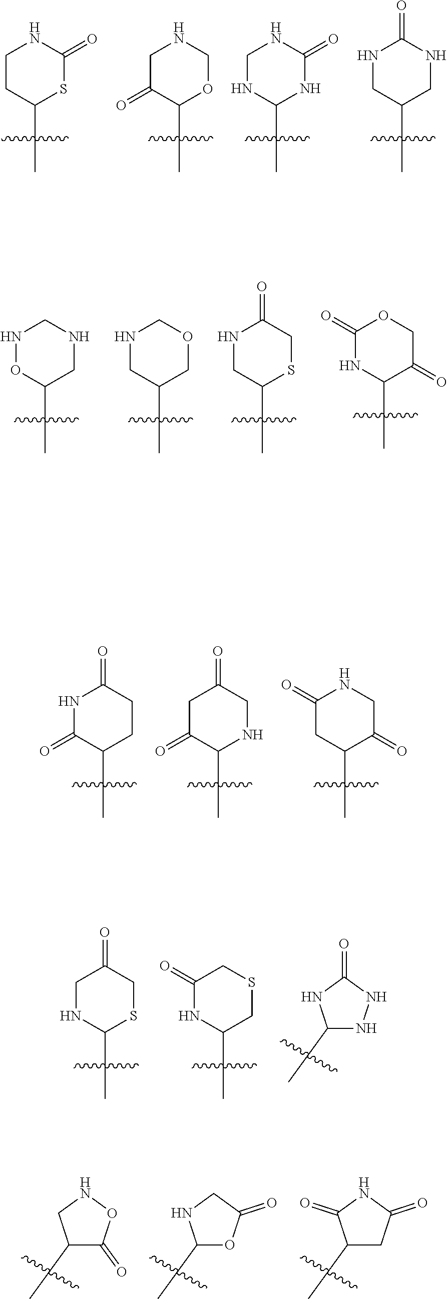 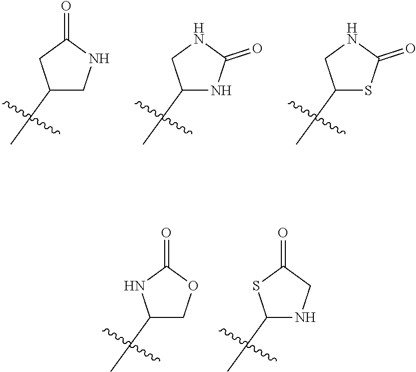 [SMILES: *C1CNC(=O)C1 ; *C1CNC(=O)N1 ; *C1CNC(=O)S1 ; *C1COC(=O)N1 ; *C1NCC(=O)S1]
ring C is an aromatic ring selected from the following structures:

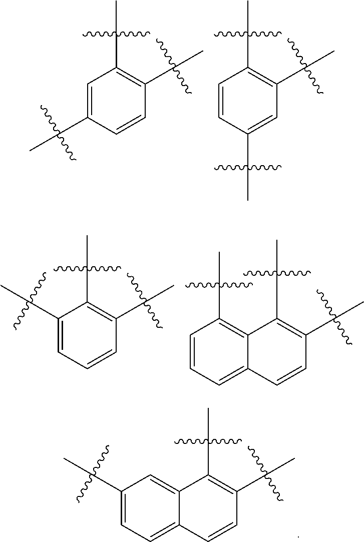 [SMILES: *c1ccc(*)c(*)c1 ; *c1ccc(*)c(*)c1 ; *c1cccc(*)c1* ; *c1ccc2cccc(*)c2c1* ; *c1ccc2ccc(*)c(*)c2c1]

18. The compound according to claim 1 or an isomer or pharmaceutically acceptable salt or deuteride thereof, wherein said compound is selected from one of the structures shown below:

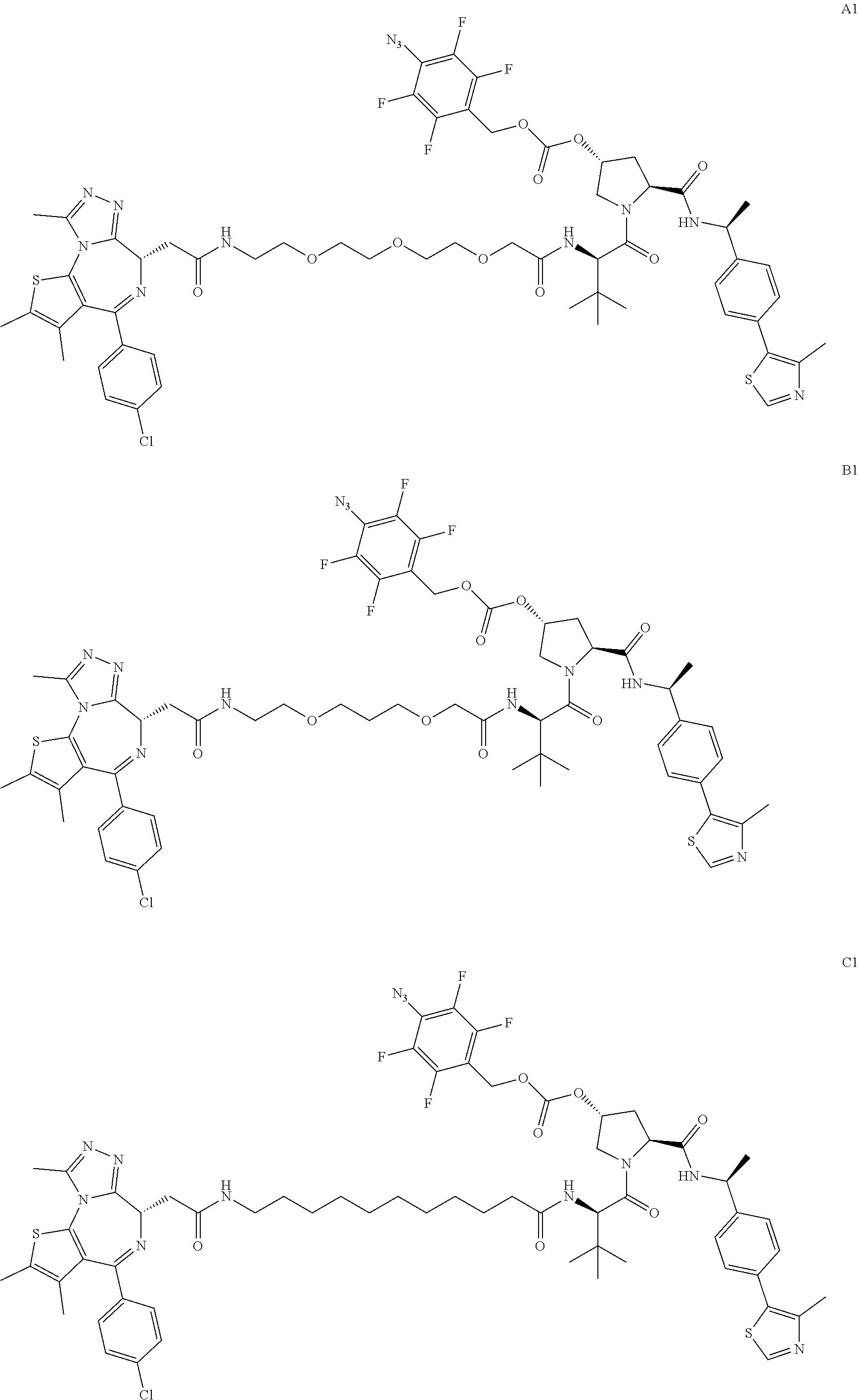

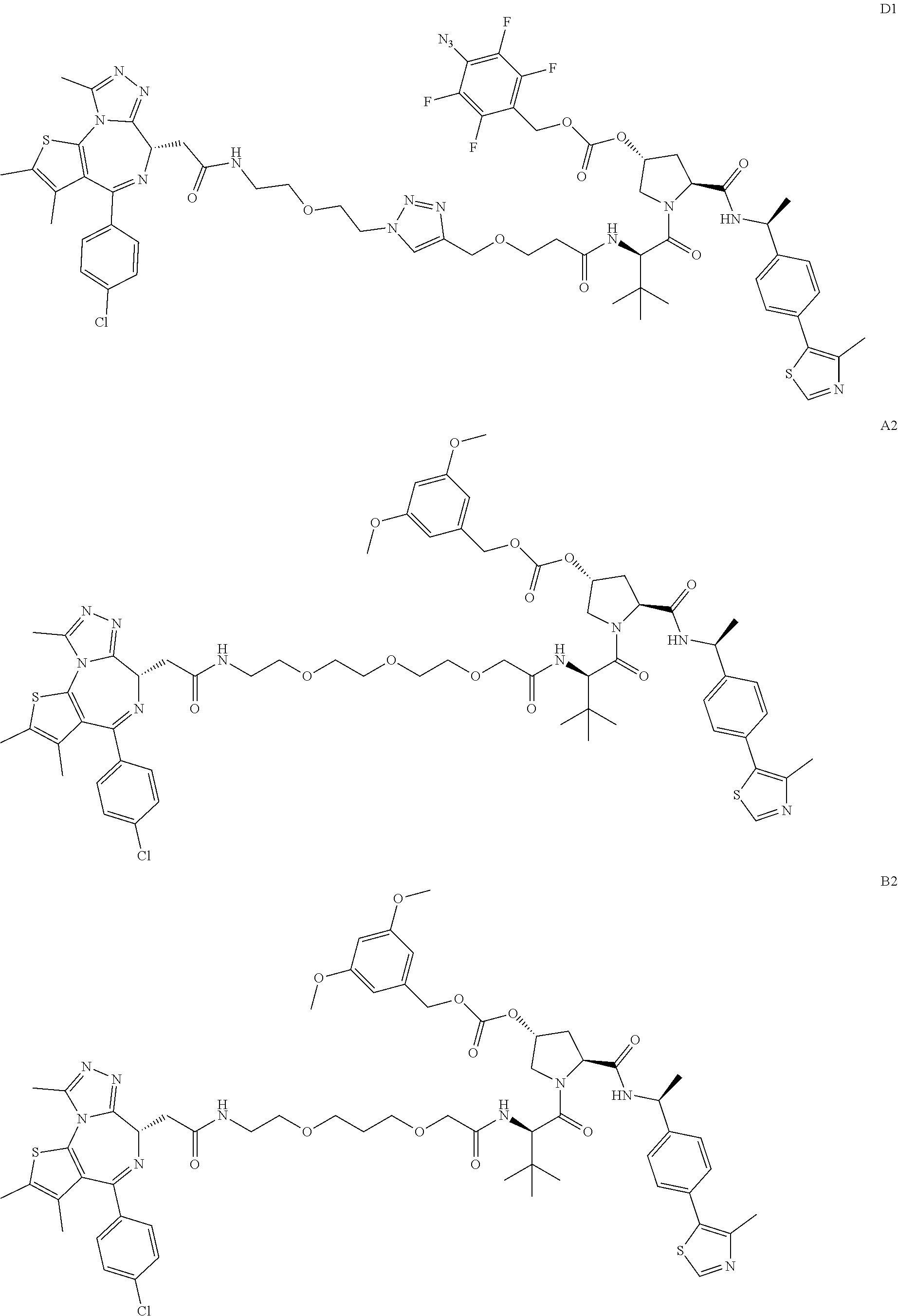

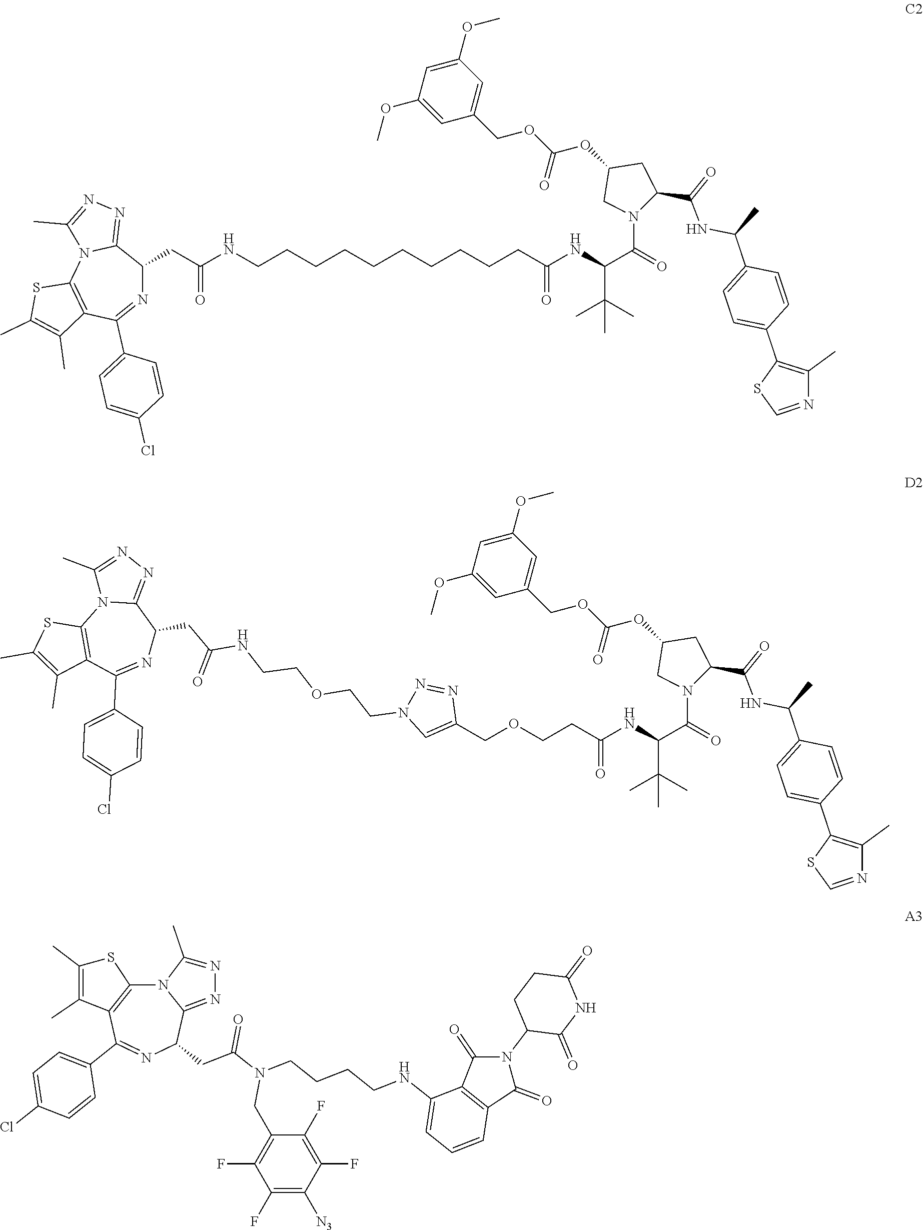

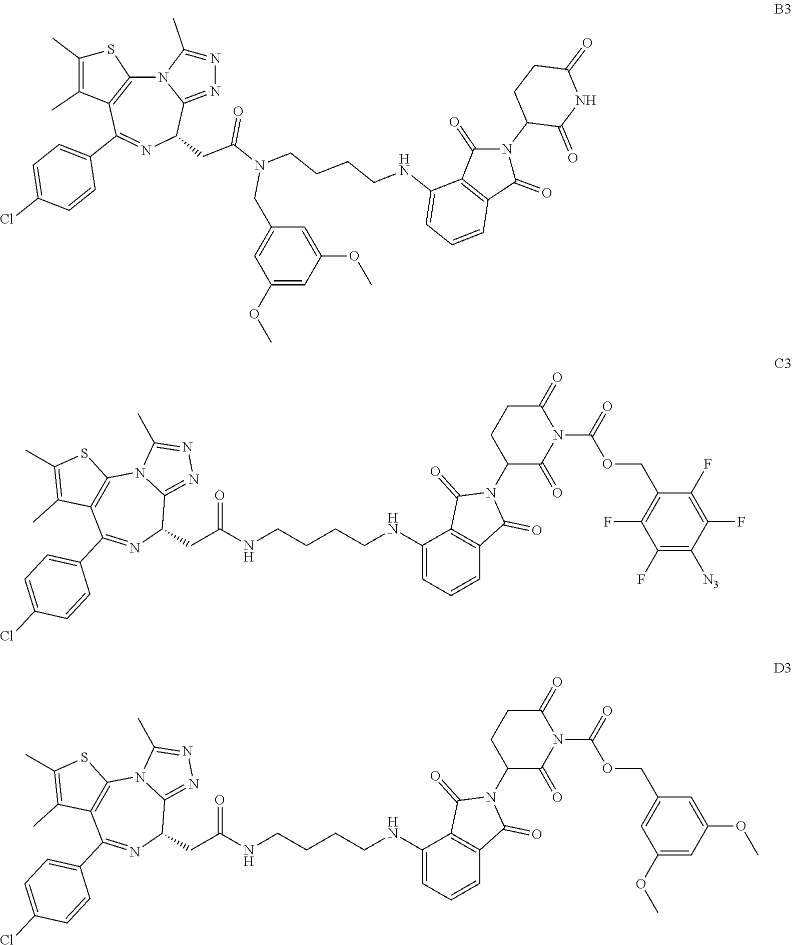 [SMILES: COc1cc(CN(CCCCNc2cccc3c2C(=O)N(C2CCC(=O)NC2=O)C3=O)C(=O)C[C@@H]2N=C(c3ccc(Cl)cc3)c3c(sc(C)c3C)-n3c(C)nnc32)cc(OC)c1 ; Cc1sc2c(c1C)C(c1ccc(Cl)cc1)=N[C@@H](CC(=O)NCCCCNc1cccc3c1C(=O)N(C1CCC(=O)N(C(=O)OCc4c(F)c(F)c(N=[N+]=[N-])c(F)c4F)C1=O)C3=O)c1nnc(C)n1-2 ; COc1cc(COC(=O)N2C(=O)CCC(N3C(=O)c4cccc(NCCCCNC(=O)C[C@@H]5N=C(c6ccc(Cl)cc6)c6c(sc(C)c6C)-n6c(C)nnc65)c4C3=O)C2=O)cc(OC)c1]

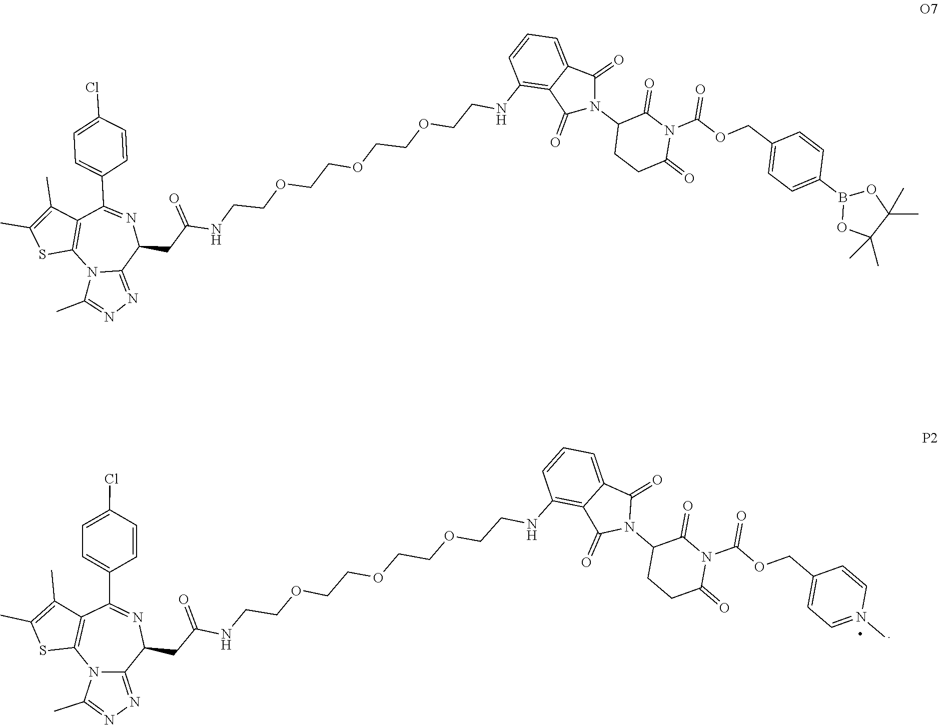

19. A compound shown in formula (VI) or an isomer or a pharmaceutically acceptable salt or deuteride thereof: 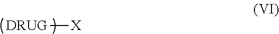
wherein X has the structure shown in claim 1.

20. The compound or isomer or pharmaceutically acceptable salt or deuterium substitute thereof according to claim 19, wherein DRUG is any PROTAC drug.

21. The compound or isomer or pharmaceutically acceptable salt or deuterium substitute thereof according to claim 19, wherein DRUG is of the following formula (VII) 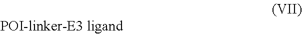
wherein the POI is selected from any target protein ligand of interest, the linker is a functional group linking the target protein ligand and the E3 ubiquitin ligase ligand, and E3 ligand is an E3 ubiquitin ligase ligand.

22. A pharmaceutical composition comprising a compound or an isomer thereof or a pharmaceutically acceptable salt or deuterium substitute as claimed in claim 1.

23. (canceled)

24. (canceled)

25. A method for treating tumors, comprising administering to a subject the compound according to claim 1, or an isomer or pharmaceutically acceptable salt or deuterated derivative thereof.

26. The method according to claim 25, wherein the method comprises administering during radiotherapy of the tumor.
